(19) United States
(12) Patent Application Publication
(10) Pub. No.: US 2010/0025669 A1
(43) Pub. Date: Feb. 4, 2010

(54) AMINE-BASED COMPOUND, ORGANIC LIGHT EMITTING DEVICE COMPRISING THE AMINE-BASED COMPOUND, AND FLAT PANEL DISPLAY DEVICE INCLUDING THE ORGANIC LIGHT EMITTING DEVICE

(76) Inventors: Seok-Hwan Hwang, Suwon-si (KR); Yoon-Hyun Kwak, Suwon-si (KR); Young-Kook Kim, Suwon-si (KR); Jeoung-In Yi, Suwon-si (KR); Jong-Hyuk Lee, Suwon-si (KR)

Correspondence Address: CHRISTIE, PARKER & HALE, LLP, PO BOX 7068, PASADENA, CA, 91109-7068, US

(21) Appl. No.: 12/511,412

(22) Filed: Jul. 29, 2009

(30) Foreign Application Priority Data
Jul. 30, 2008 (KR) 10-2008-0074713

(51) Int. Cl. H01L 51/54; C07D 209/86; C07D 401/12

(52) U.S. Cl. 257/40; 548/445; 546/276.7; 257/98; 257/E51.047

(57) ABSTRACT

The invention is directed to an amine-based compound represented by Formula 1, an organic light emitting device with an organic film including the same, and a flat panel display device including the organic light emitting device.

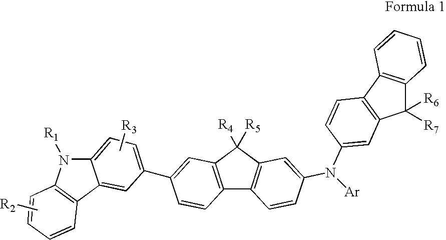

### CROSS-REFERENCE TO RELATED PATENT APPLICATION

This application claims priority to and the benefit of Korean Patent Application No. 10-2008-0074713, filed on Jul. 30, 2008 in the Korean Intellectual Property Office, the entire content of which is incorporated herein by reference.

### BACKGROUND OF THE INVENTION

1. Field of the Invention

The present invention relates to amine-based compounds and organic light emitting devices with an organic film including the same.

2. Description of the Related Art

Organic light emitting diodes are self light-emitting devices that have wide viewing angles, excellent contrast, and quick response time, and thus have received a lot of attention. In addition, organic light emitting diodes have high luminance, low operating voltage, and can display a full range of color. Therefore, research in organic light emitting diodes continues to grow.

Organic light emitting diodes conventionally have an anode/organic layer/cathode structure. The organic layer may include at least one of a hole injection layer, a hole transport layer, an emissive layer, an electron transport layer, and an electron injection layer.

Materials for forming the hole injection layer and the hole transport layer include polyphenyl compounds, and anthracene derivatives. However, organic light emitting diodes including a hole injection layer and/or a hole transport layer formed from conventional materials do hot have satisfactory life span, efficiency, and power consumption, thus leaving room for improvement.

### SUMMARY OF THE INVENTION

According to an embodiment of the present invention, an amine-based compound represented by Formula 1 is provided.

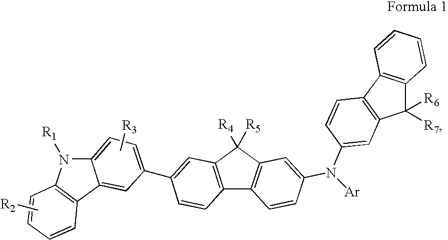

where Ar is a substituted or unsubstituted C₆-C₃₀ aryl group, a substituted or unsubstituted C₆-C₂₀ aryloxy group, a substituted or unsubstituted C₄-C₂₀ heteroaryl group, or a substituted or unsubstituted C₄-C₂₀ condensed polycyclic group, R₁ is hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ heteroaryl group, or a substituted or unsubstituted C₄-C₂₀ condensed polycyclic group,

each of R₂ and R₃ is independently hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ condensed polycyclic group, fluorine, a cyano group, or an amino group, and

each of R₄, R₅, R₆, and R₇ is independently hydrogen, a substituted or unsubstituted C₁-C₂₀ alkyl group, a substituted or unsubstituted C₁-C₂₀ alkoxy group, or a substituted or unsubstituted C₆-C₃₀ aryl group.

According to another embodiment of the present invention, an organic light emitting device includes a first electrode, a second electrode, and an organic film interposed between the first electrode and the second electrode. In one embodiment, the organic film includes the amine-based compound.

The organic layer may be a hole injection layer, a hole transport layer, or an emissive layer.

According to another embodiment of the present invention, a flat panel display device is provided. The flat panel display device includes the organic light emitting device and a thin-film transistor, where the electrode of the organic light emitting device is electrically connected to a source electrode or a drain electrode of the thin-film transistor.

### BRIEF DESCRIPTION OF THE DRAWINGS

These and other features of the present invention will be better understood by reference to the following detailed description when considered in conjunction with the attached drawings in which:

FIG. 1 is a diagram illustrating a structure of an organic light emitting device according to an embodiment of the present invention; and

FIG. 2 is a graph comparing the life spans of an organic light emitting device according to an embodiment of the present invention and a conventional organic light emitting device.

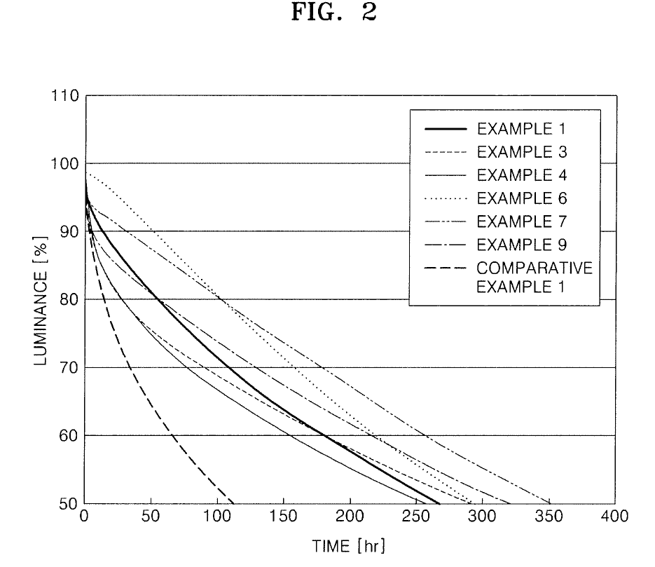

### DETAILED DESCRIPTION OF THE INVENTION

The present invention will now be described in detail with reference to the accompanying drawings, in which exemplary embodiments of the invention are shown.

An amine-based compound represented by Formula 1 below has a carbazolyl group and a fluorenyl group.

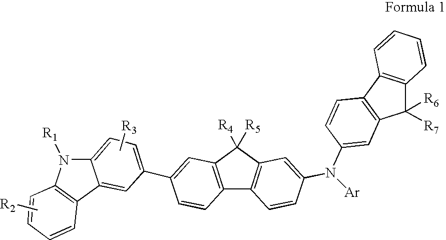

The amine-based compound of Formula 1 having both a carbazolyl group and a fluorenyl group may function as a hole injecting material, a hole transporting material, and/or a light emitting material.

The amine-based compound of Formula 1 has a high glass transition temperature (Tg) or melting point due to the presence of the carbazolyl group. As a result, it is highly resistant to Joule's heat produced inside the organic layers, between the organic layers or between an organic layer and a metal electrode. Therefore, an organic light emitting device including the amine-based compound of Formula 1 is highly durable under all kind of conditions, including manufacturing, storing, and operating conditions.

In addition, the presence of the fluorenyl group in the amine-based compound of Formula 1 further improves the film properties. As a result, organic light emitting devices with an organic film having the amine-based compound of Formula 1 have improved performance and overall film characteristics.

In Formula 1, Ar may be a substituted or unsubstituted C₆-C₃₀ aryl group, a substituted or unsubstituted C₆-C₂₀ aryloxy group, a substituted or unsubstituted C₄-C₂₀ heteroaryl group, or a substituted or unsubstituted C₄-C₂₀ condensed polycyclic group.

In particular, Ar may be a phenyl group, a biphenyl group, a terphenyl group, a naphthyl group, a pentalenyl group, an indenyl group, an anthracenyl group, an azulenyl group, a heptalenyl group, an acenaphthylenyl group, a fluorenyl group, a penanthrenyl group, a triphenylenyl group, a naphthacenyl group, a phenalenyl group, a pyrenyl group, a pentaphenyl group, a hexaphenyl group, a pyridinyl group, a quinolinyl group, a pyrazinyl group, a pyrimidinyl group, or a carbazolyl group; all of which mentioned groups may be unsubstituted or substituted with a substituent. The substituent may be selected from halogen atoms, cyano groups, amino groups, (C₁-C₁₀ alkyl)amino groups, di(C₁-C₁₀ alkyl)amino groups, (C₆-C₁₄ aryl)amino groups, di(C₆-C₁₄ aryl)amino groups, C₁-C₁₀ alkyl groups, C₁-C₁₀ alkoxy groups, C₆-C₁₄ aryloxy groups, C₆-C₁₄ aryl groups, and C₆-C₁₄ aryl groups substituted with a C₆-C₁₄ aryl substituent. However, Ar is not limited thereto.

Other suitable materials for Ar include phenyl groups, lower alkyl phenyl groups, lower alkoxy phenyl groups, cyanophenyl groups, phenoxyphenyl groups, fluorophenyl groups, naphthyl groups, lower alkyl naphthyl groups, lower alkoxy naphthyl groups, cyanonaphthyl groups, halonaphthyl groups, fluorenyl groups, carbazolyl groups, lower alkyl carbazolyl groups, biphenyl groups, lower alkyl biphenyl groups, lower alkoxy biphenyl groups, terphenyl groups, pyridyl groups, and quinolinyl groups. The lower alkyl and lower alkoxy groups may have a carbon number ranging from 1 to 5.

Other nonlimiting examples of suitable materials for Ar may include phenyl groups, ethylphenyl groups, ethylbiphenyl groups, o-, m-, or p-fluorophenyl groups, dichlorophenyl groups, cyanophenyl groups, trifluorophenyl groups, methoxyphenyl groups, o-, m-, or p-tolyl groups, mesityl groups, phenoxyphenyl groups, (α, α-dimethyl benzene)phenyl groups, (N,N′-dimethyl)aminophenyl groups, (N,N′-diphenyl)aminophenyl groups, pentalenyl groups, naphthyl groups, methylnaphthyl groups, anthracenyl groups, azulenyl groups, heptalenyl groups, acenaphthylenyl groups, fluorenyl groups, anthraquinolyl groups, phenanthryl groups, triphenylene groups, naphthacenyl groups, pyrenyl groups, biphenyl groups, methylbiphenyl groups, pentaphenyl groups, hexaphenyl groups, carbazolyl groups, pyridyl groups, and quinolyl groups.

According to certain embodiments, Ar may be a monocyclic, bicyclic, or tricyclic aryl group selected from phenyl groups, naphthyl groups, biphenyl groups, terphenyl groups, fluorenyl groups, and carbazolyl groups, and monocyclic, bicyclic, and tricyclic aryl groups thereof substituted with 1 to 3 substituents selected from C₁-C₅ alkyl groups, C₁-C₅ alkoxy groups, cyano groups, amino groups, phenoxy groups, phenyl groups, and halogen atoms.

According to still further embodiments, Ar may be any one of the groups represented by Formulae 2a through 2q below, but is not limited thereto:

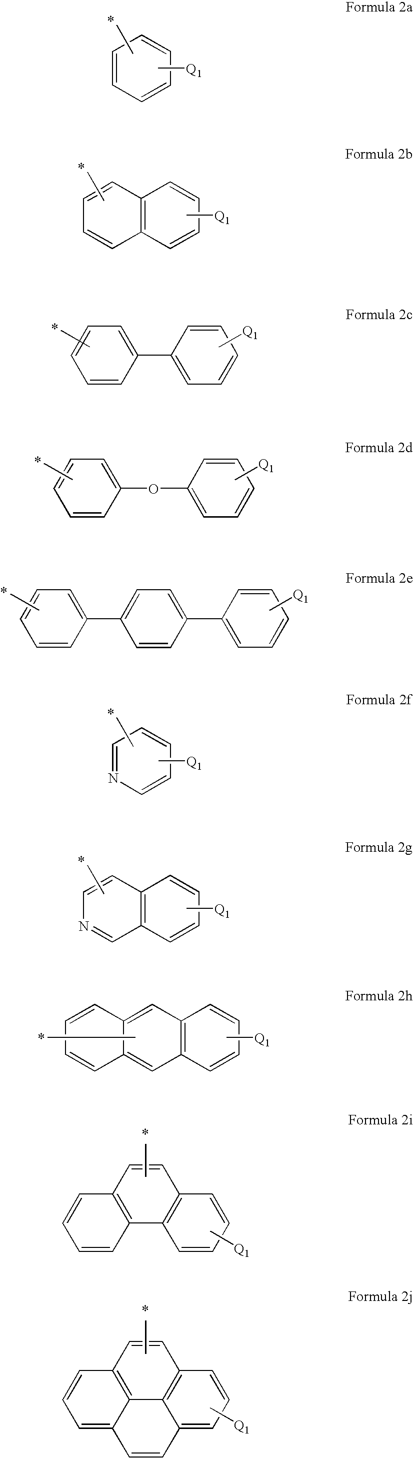

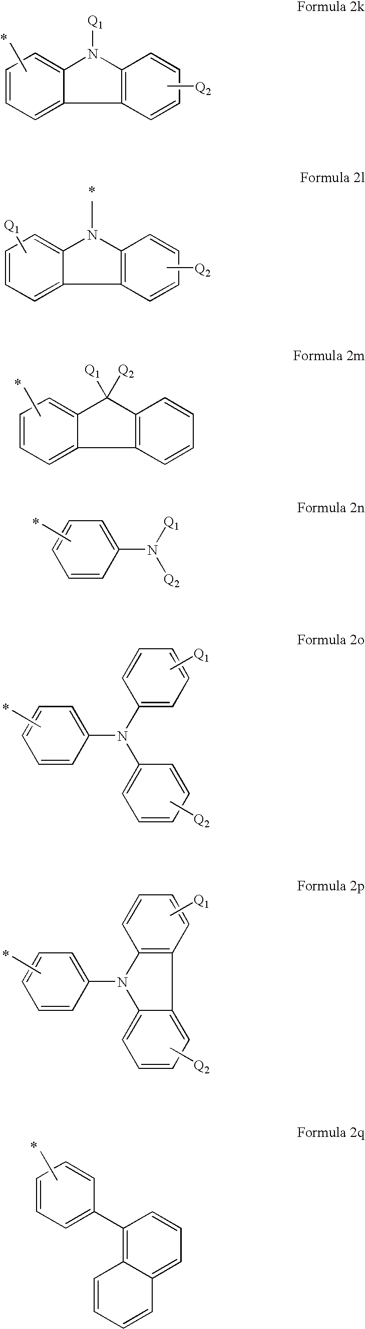

Each of Q₁ and Q₂ in Formulae 2a through 2q may independently be hydrogen, fluorine, a cyano group, an amino group, a C₁-C₁₀ alkyl group, a C₁-C₁₀ alkoxy group, a C₆-C₁₄ aryl group, or a C₆-C₁₄ aryl group substituted with a C₆-C₁₄ aryl substituent. The symbol, *, in Formulae 2a through 2q represents a binding site with N of Formula 1. The C₆-C₁₄ aryl group substituted with a C₆-C₁₄ aryl substituent may be, for example, a biphenyl group or terphenyl group substituted with a C₁-C₁₀ alkyl substituent, or a C₁-C₁₀ alkoxy group.

In one embodiment, Ar is a group represented by Formula 2a, 2b, 2c, or 2h.

In Formula 1, R₁ may be hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ heteroaryl group, or a substituted or unsubstituted C₄-C₂₀ condensed polycyclic group.

In particular, R₁ may be a substituted or unsubstituted C₆-C₁₄ aryl group.

More particularly, R₁ may be a phenyl group, a naphthyl group, a pentalenyl group, an indenyl group, an anthracenyl group, an azulenyl group, a heptalenyl group, an acenaphthylenyl group, a fluorenyl group, a penanthrenyl group, a triphenylenyl group, a naphthacenyl group, a phenalenyl group, a pyrenyl group, a pentaphenyl group, or a hexaphenyl group, all of which mentioned groups may be unsubstituted or substituted with a substituent. The substituent can be selected from halogen atoms, cyano groups, amino groups, (C₁-C₁₀ alkyl)amino groups, di(C₁-C₁₀ alkyl)amino groups, (C₆-C₁₄ aryl)amino groups, di(C₆-C₁₄ aryl)amino groups, C₁-C₁₀ alkyl groups, C₁-C₁₀ alkoxy groups, C₆-C₁₄ aryloxy groups, C₆-C₁₄ aryl groups, and C₆-C₁₄ aryl groups substituted with a C₆-C₁₄ aryl substituent. However, R₁ is not limited thereto.

In one embodiment, R₁ may be a phenyl group, a naphthyl group, a halophenyl group, or a phenyl group substituted with a phenyl substituent (biphenyl group).

In Formula 1, each of R₂ and R₃ may independently be hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ condensed polycyclic group, fluorine, a cyano group, or an amino group.

In particular, each of R₂ and R₃ may independently be hydrogen, fluorine, a cyano group, an amino group, a C₁-C₁₀ alkyl group, or a C₁-C₁₀ alkoxy group. More particularly, R₂ and R₃ can be both hydrogen.

In Formula 1, each of R₄, R₅, R₆, and R₇ may independently be hydrogen, a substituted or unsubstituted C₁-C₂₀ alkyl group, a substituted or unsubstituted C₁-C₂₀ alkoxy group, or a substituted or unsubstituted C₆-C₃₀ aryl group.

In particular, each of R₄ through R₇ may independently be hydrogen, a C₁-C₁₀ alkyl group, a C₁-C₁₀ alkoxy group, or a C₆-C₁₄ aryl group. More particularly, each of R₄ through R₇ may independently be hydrogen, a C₁-C₁₀ alkyl group, a phenyl group, or a naphthyl group.

The unsubstituted C₁-C₂₀ alkyl group may be linear or branched. Nonlimiting examples of suitable unsubstituted C₁-C₂₀ alkyl groups include methyl groups, ethyl groups, propyl groups, isobutyl groups, sec-butyl groups, pentyl groups, iso-amyl groups, hexyl groups, heptyl groups, octyl groups, nonyl groups, and dodecyl groups. At least one hydrogen atom of the alkyl group may be substituted with a halogen, a hydroxyl group, a nitro group, a cyano group, an amino group, an amidino group, hydrazine, hydrazone, a carboxyl group or a salt thereof, a sulfonic acid group or a salt thereof, a phosphoric acid group or a salt thereof, a C₁-C₁₀ alkyl group, a C₁-C₁₀ alkoxy group, a C₂-C₁₀ alkenyl group, a C₂-C₁₀ alkynyl group, a C₆-C₁₆ aryl group, or a C₄-C₁₆ heteroaryl group.

The unsubstituted C₁-C₂₀ alkoxy group is a group that may have a —OA structure, where A is an unsubstituted C₁-C₂₀ alkyl group as described above. Nonlimiting examples of suitable unsubstituted C₁-C₂₀ alkoxy groups include methoxy groups, ethoxy groups, propoxy groups, isopropoxy groups, butoxy groups, and pentoxy groups. At least one hydrogen atom of the alkoxy group may be substituted with the same substituent groups as previously described for the alkyl groups.

In one embodiment, the unsubstituted C₆-C₃₀ aryl group is a C₆-C₃₀ carbocyclic aromatic system containing at least one ring. In a two-ring system, at least two rings can be fused with each other or attached to each other by single bond. In one embodiment, at least one hydrogen atom of the aryl group is substituted with the same substituent groups as previously described for the alkyl groups.

Nonlimiting examples of suitable substituted or unsubstituted C₆-C₃₀ aryl groups include phenyl groups, C₁-C₁₀ alkylphenyl groups (for example, ethyl phenyl groups), halophenyl groups (for example, o-, m- or p-fluorophenyl groups, dichlorophenyl groups), cyanophenyl groups, dicyanophenyl groups, trifluoromethoxyphenyl groups, biphenyl groups, halobiphenyl groups, cyanobiphenyl groups, C₁-C₁₀ alkyl biphenyl groups, C₁-C₁₀ alkoxybiphenyl groups, o-, m- or p-tolyl groups, o-, m- or p-cumenyl groups, mesityl groups, phenoxyphenyl groups, (α,α-dimethylbenzene)phenyl groups, (N,N′-dimethyl)aminophenyl groups, (N,N′-diphenyl)aminophenyl groups, pentalenyl groups, indenyl groups, naphthyl groups, halonaphthyl groups (for example, fluoronaphthyl groups), C₁-C₁₀ alkylnaphthyl groups (for example, methylnaphthyl groups), C₁-C₁₀ alkoxynaphthyl groups (for example, methoxynaphthyl groups), cyanonaphthyl groups, anthracenyl groups, azulenyl groups, heptalenyl groups, acenaphthylenyl groups, phenalenyl groups, fluorenyl groups, anthraquinolyl groups, methylanthryl groups, phenanthryl groups, triphenylene groups, pyrenyl groups, chrysenyl groups, ethyl-chrysenyl groups, picenyl groups, perylenyl groups, chloroperylenyl groups, pentaphenyl groups, pentacenyl groups, tetraphenylenyl groups, hexaphenyl groups, hexacenyl groups, rubicenyl groups, coroneryl groups, trinaphthylenyl groups, heptaphenyl groups, heptacenyl groups, pyranetrenyl groups, and ovalenyl groups.

In one embodiment, the unsubstituted C₆-C₃₀ aryloxy group has a formula —OB, where B is a C₆-C₃₀ aryl group as described above. Nonlimiting examples of suitable C₆-C₃₀ aryloxy groups include phenoxy groups and naphthyloxy groups. At least one hydrogen atom of the aryloxy group may be substituted with the same substituent groups as previously described for the alkyl groups.

In one embodiment, the unsubstituted C₄-C₂₀ heteroaryl group includes an atom selected from the group consisting of N, O, P, and S, and at least one aromatic ring. At least two rings may be fused with each other or attached to each other by a single bond. At least one hydrogen atom of the heteroaryl group may be substituted with the same substituent groups as previously described for the alkyl groups.

Nonlimiting examples of suitable unsubstituted C₄-C₂₀ heteroaryl groups include pyrazolyl groups, imidazolyl groups, oxazolyl groups, thiazolyl groups, triazolyl groups, tetrazolyl groups, oxadiazolyl groups, pyridyl groups, pyridazinyl groups, pyrimidinyl groups, triazinyl groups, carbazolyl groups, indolyl groups, quinolyl groups, and isoquinolyl groups. These groups may be substituted with the same substituent groups as previously described for the alkyl groups.

In one embodiment, the unsubstituted C₄-C₂₀ condensed polycyclic group is a substituent including at least two rings, where at least one aromatic ring and/or at least one non-aromatic ring are fused with each other. In another embodiment, they aryl group or heteroaryl group as described above may be included in the condensed polycyclic group.

Nonlimiting examples of suitable amine-based compounds of Formula 1 include Compounds 1 through 96 represented by Formula 3 below.

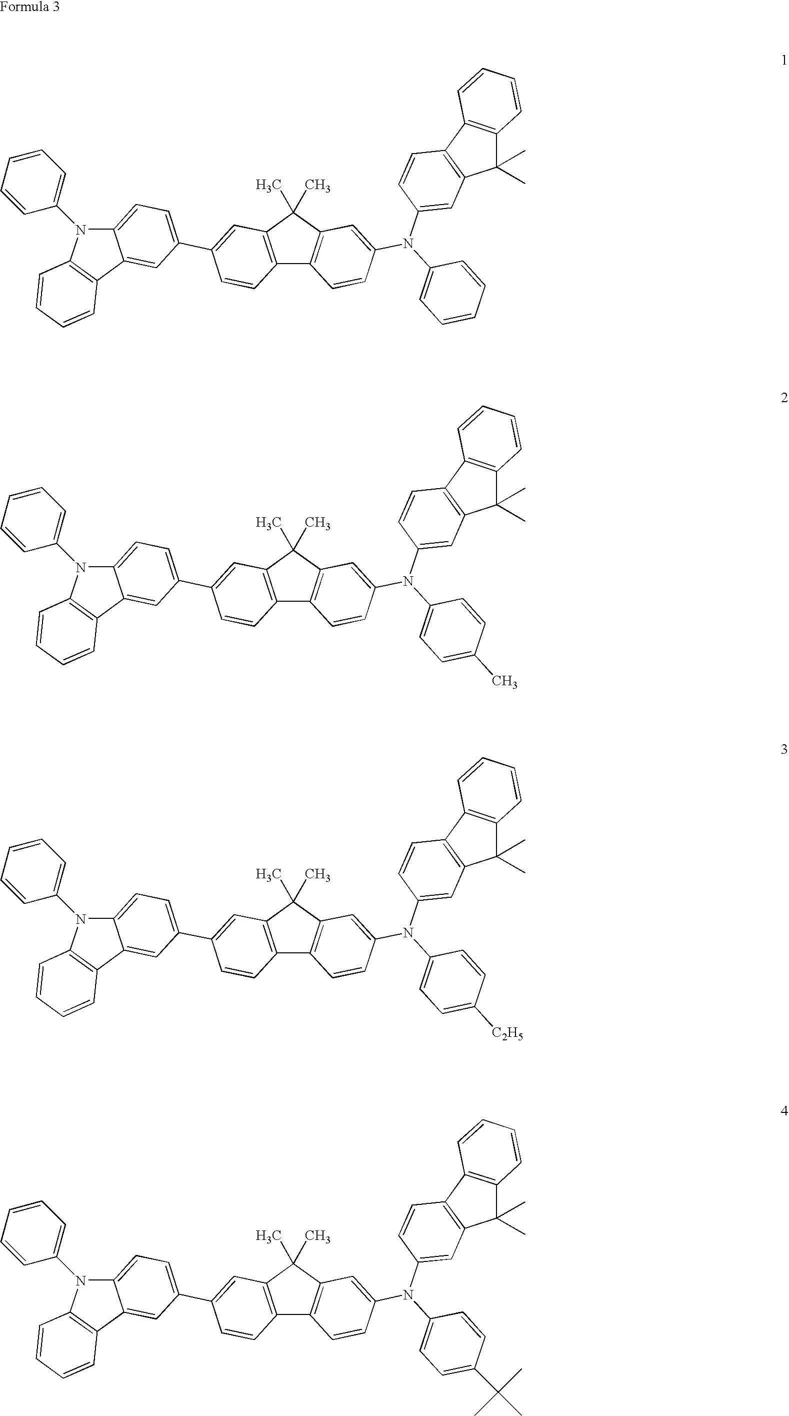

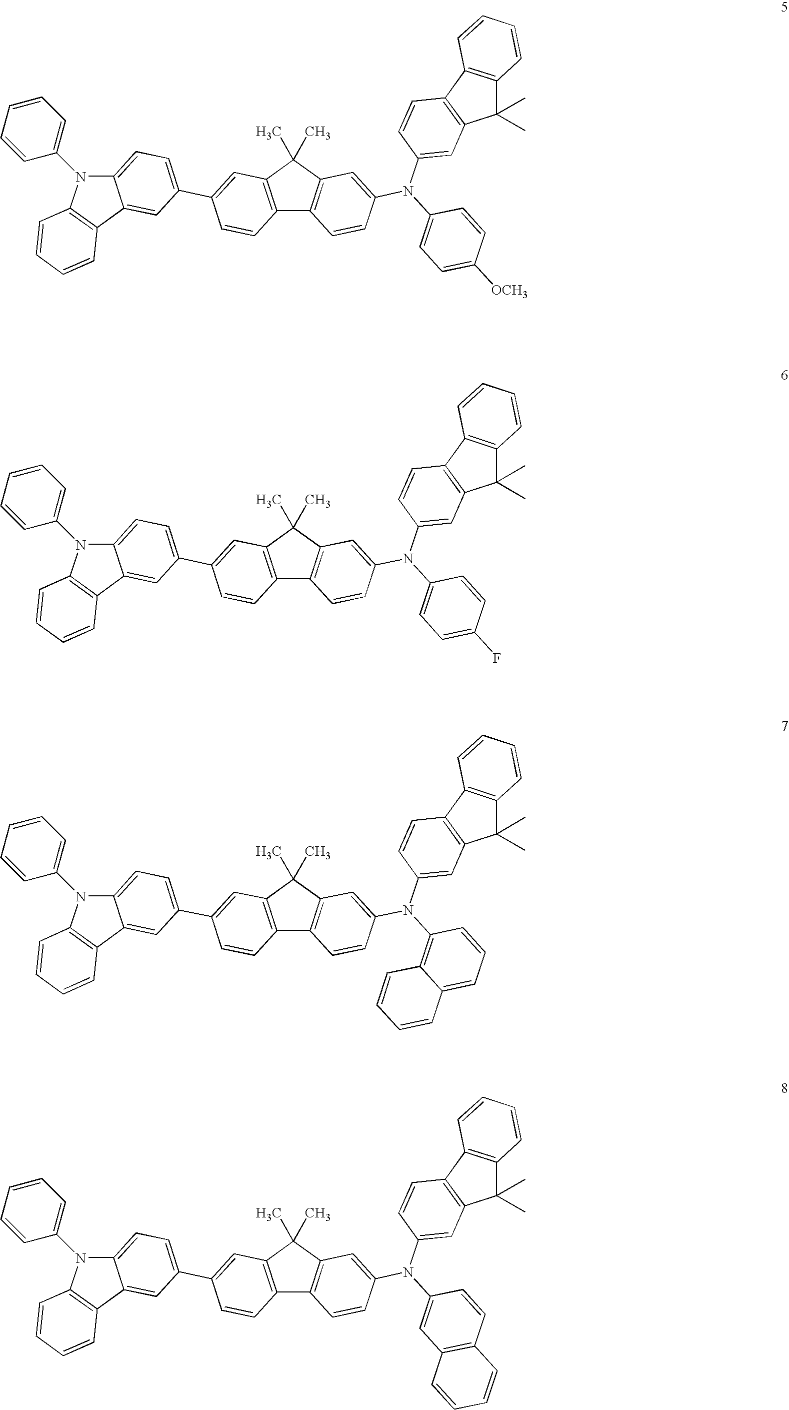

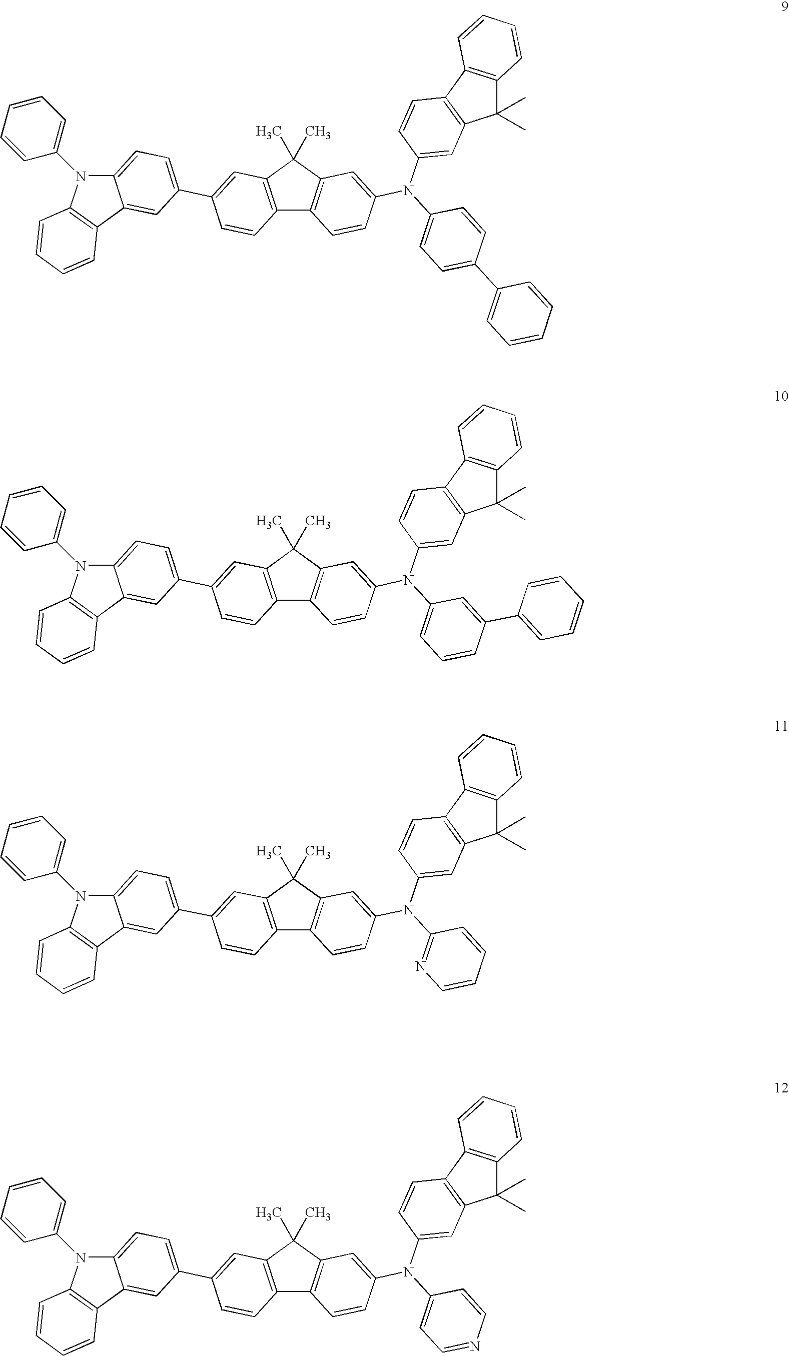

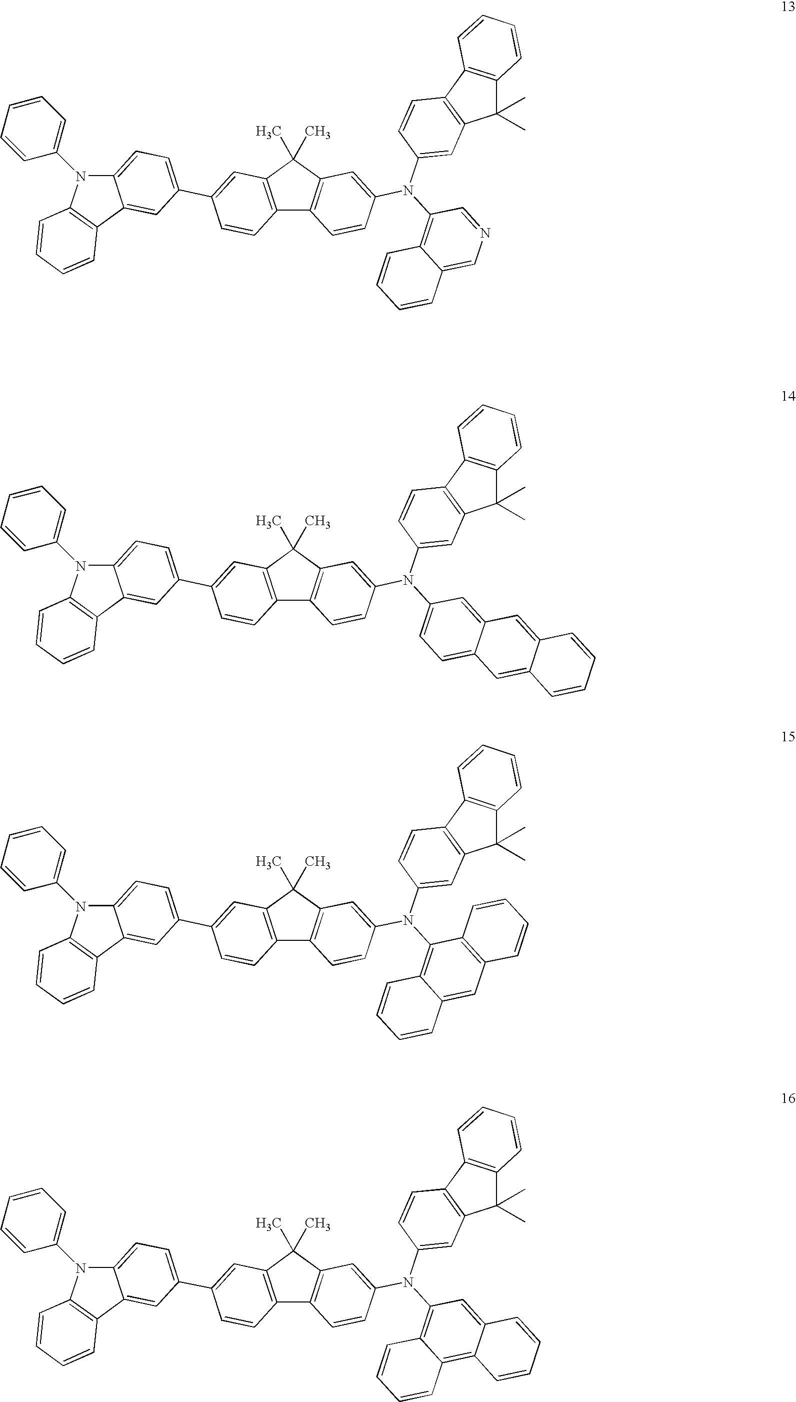

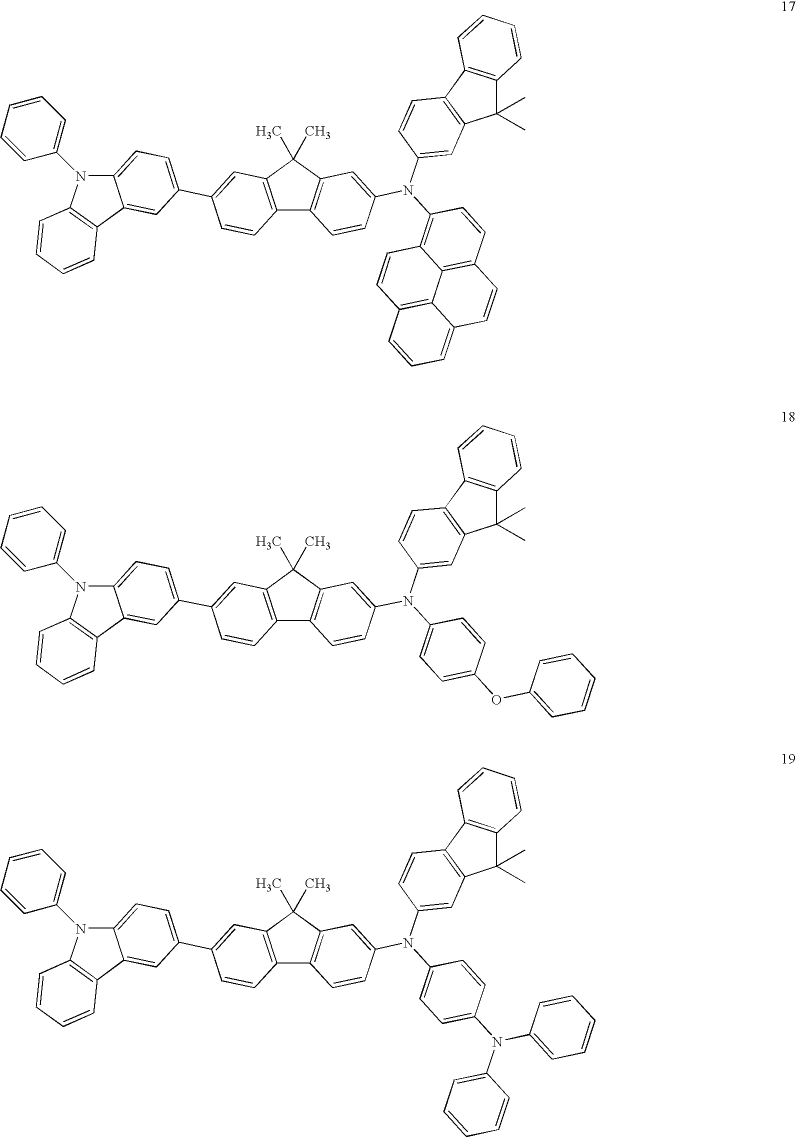

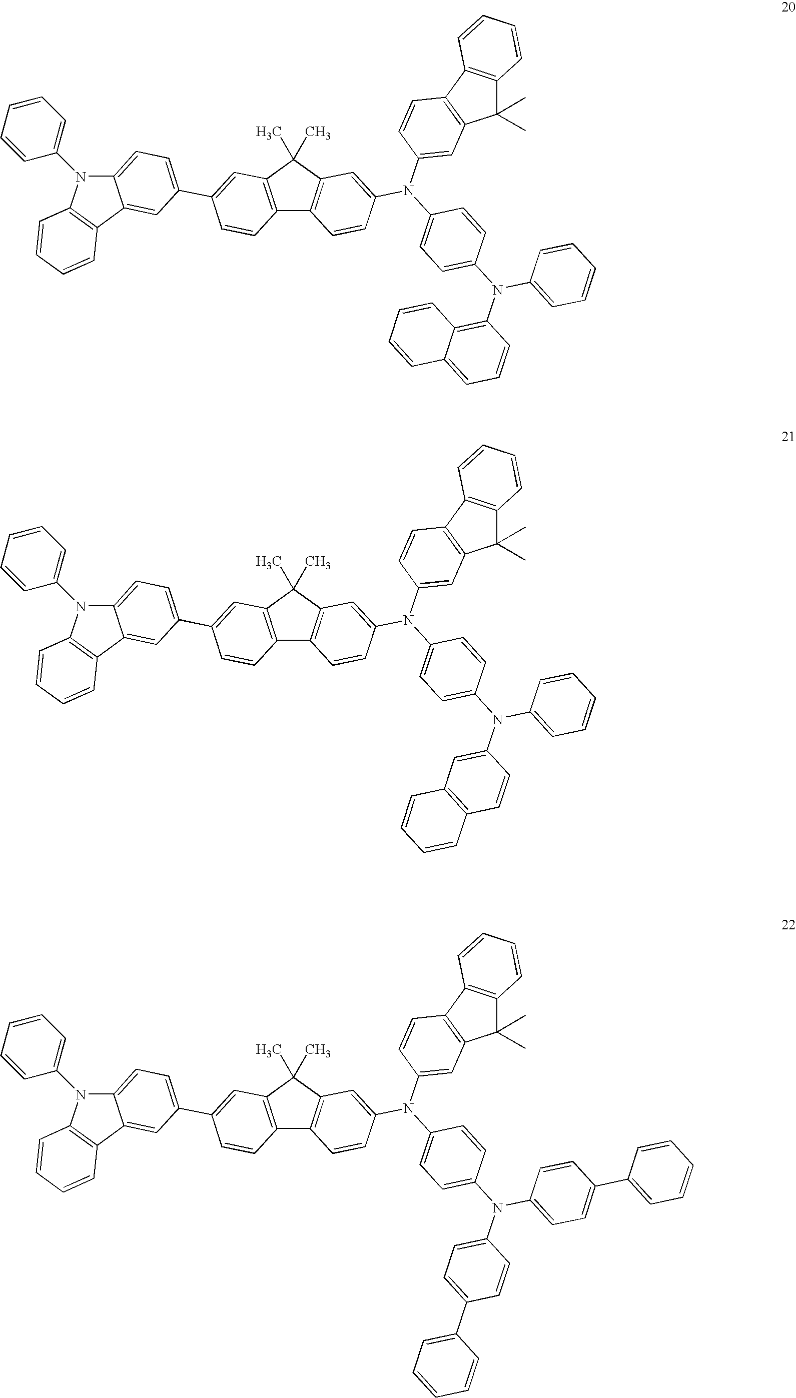

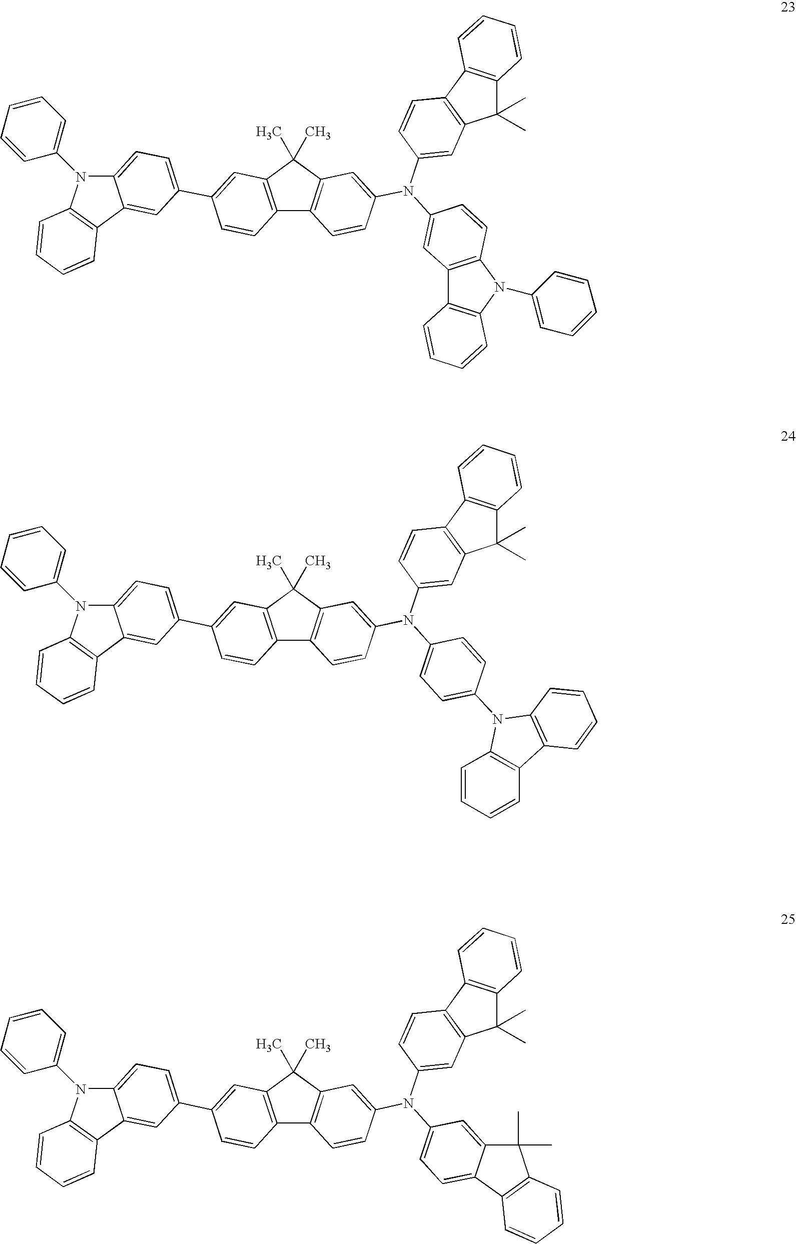

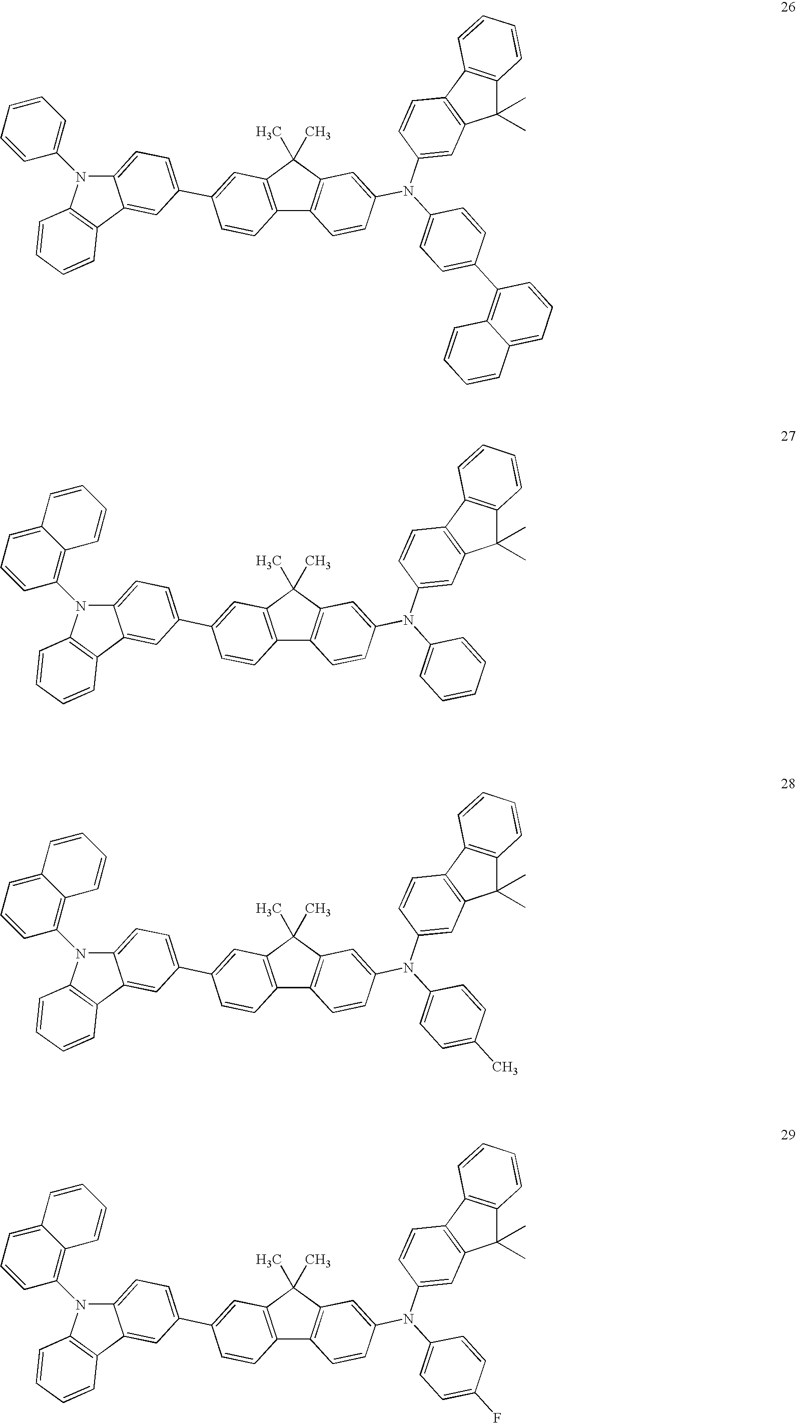

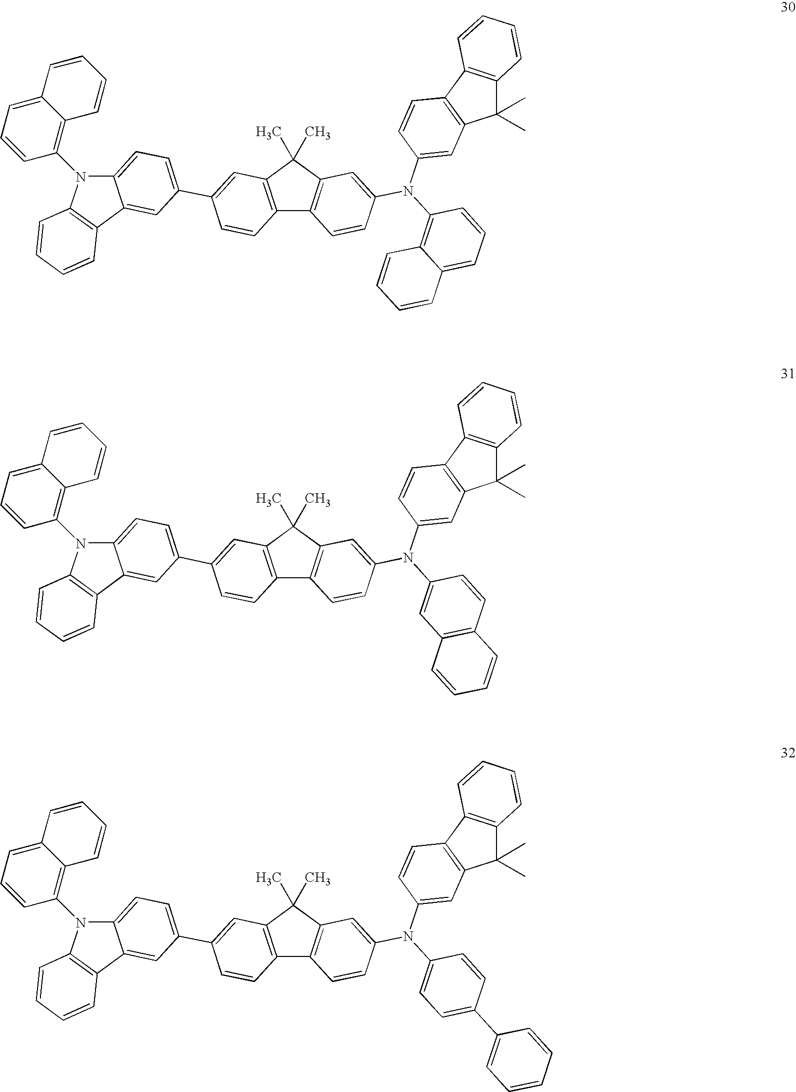

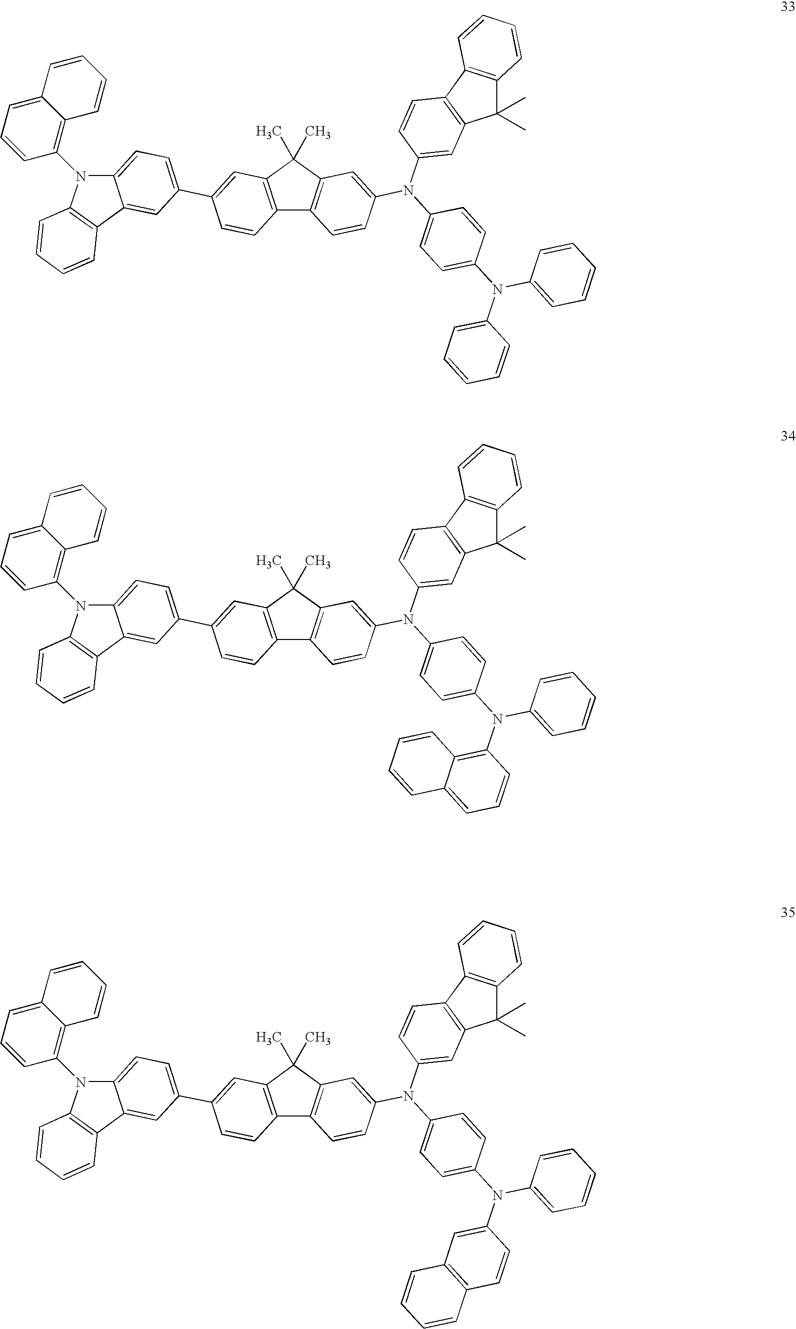

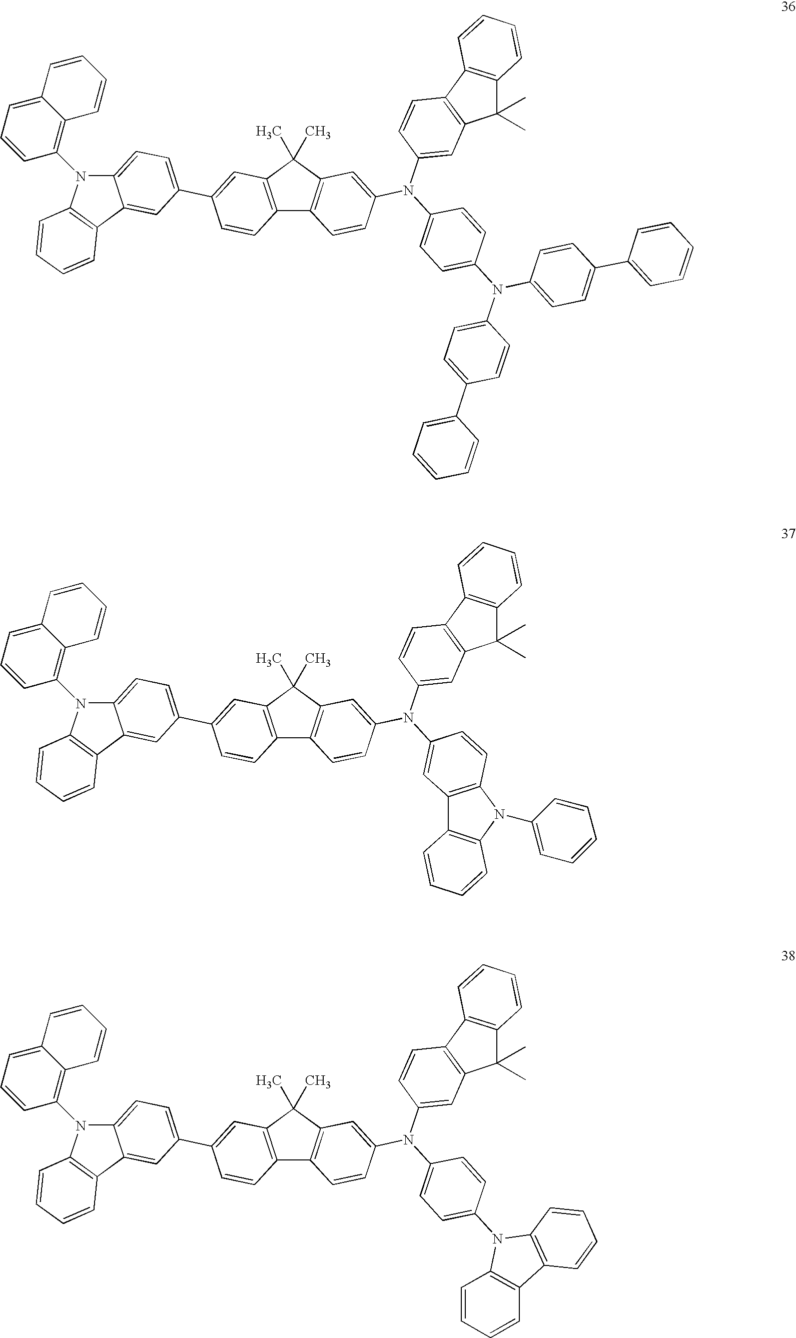

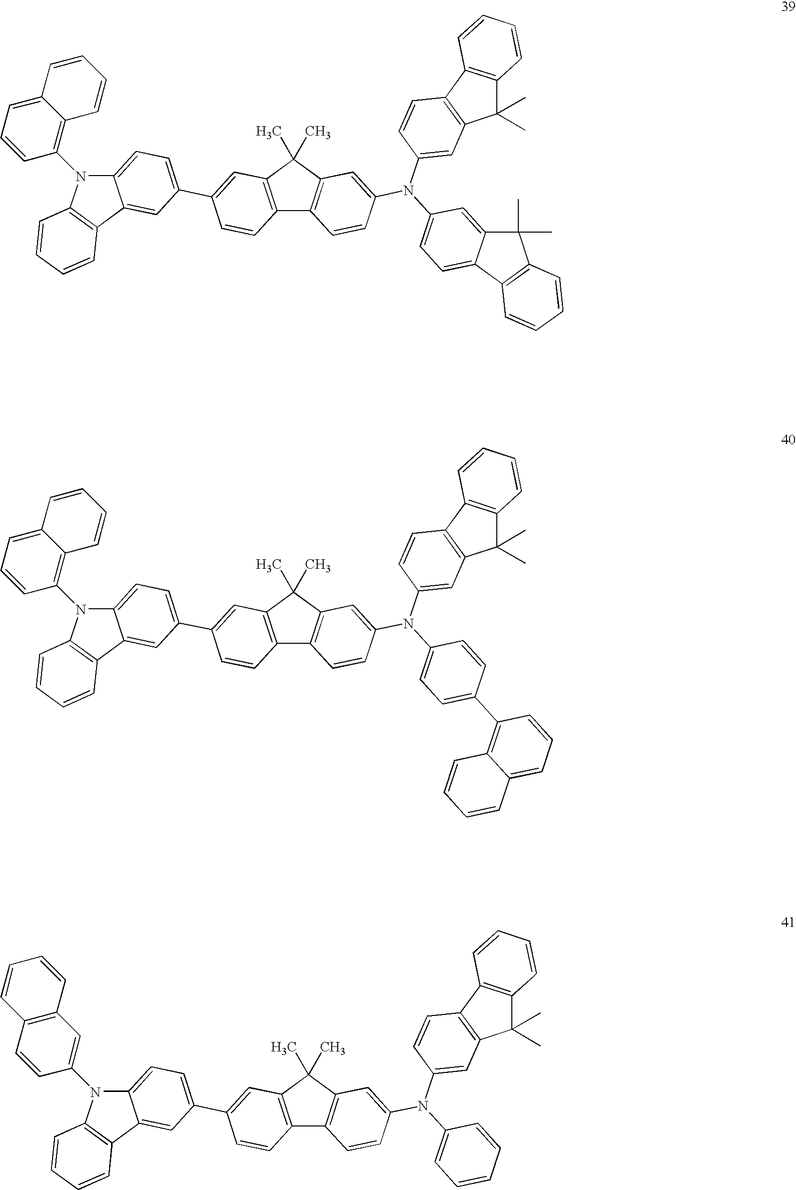

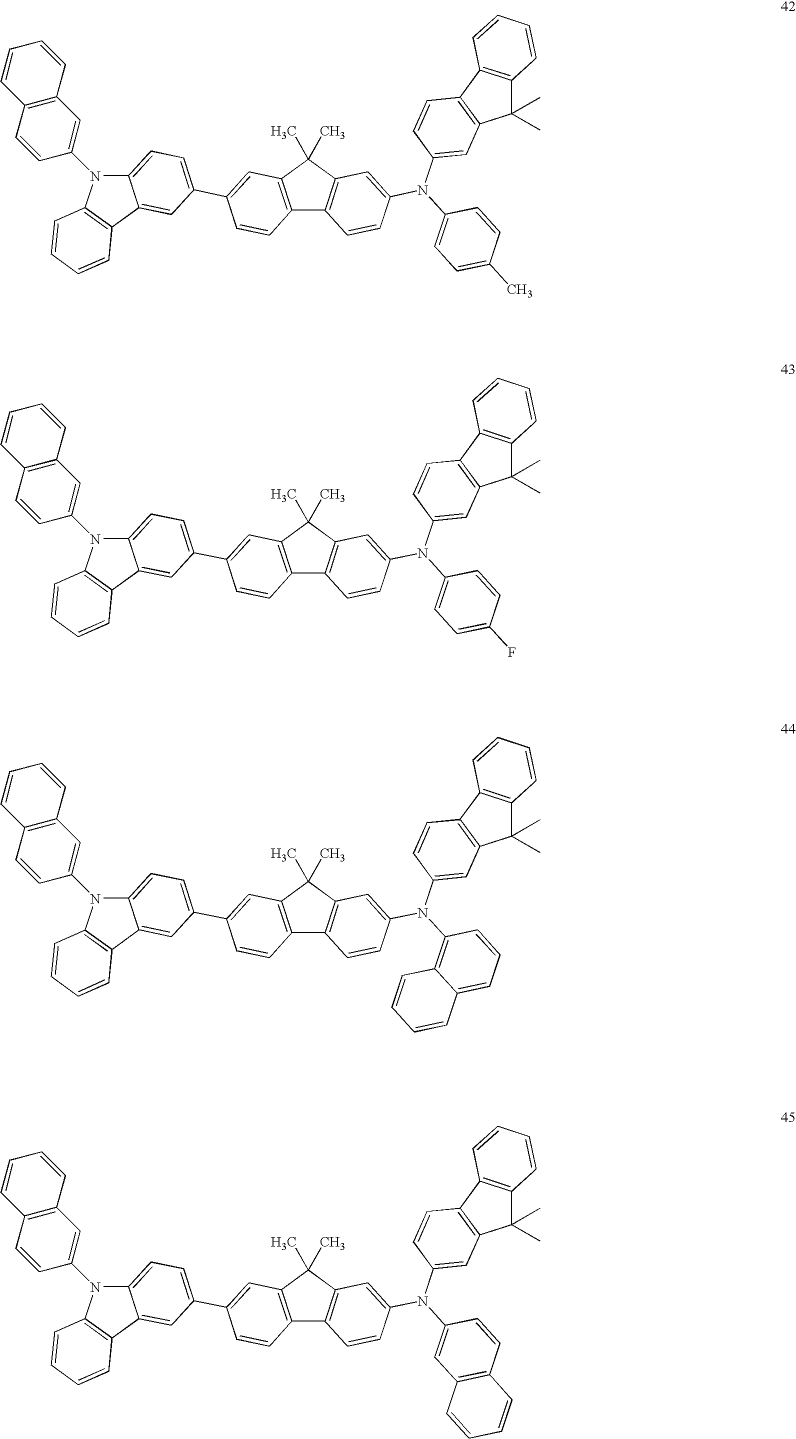

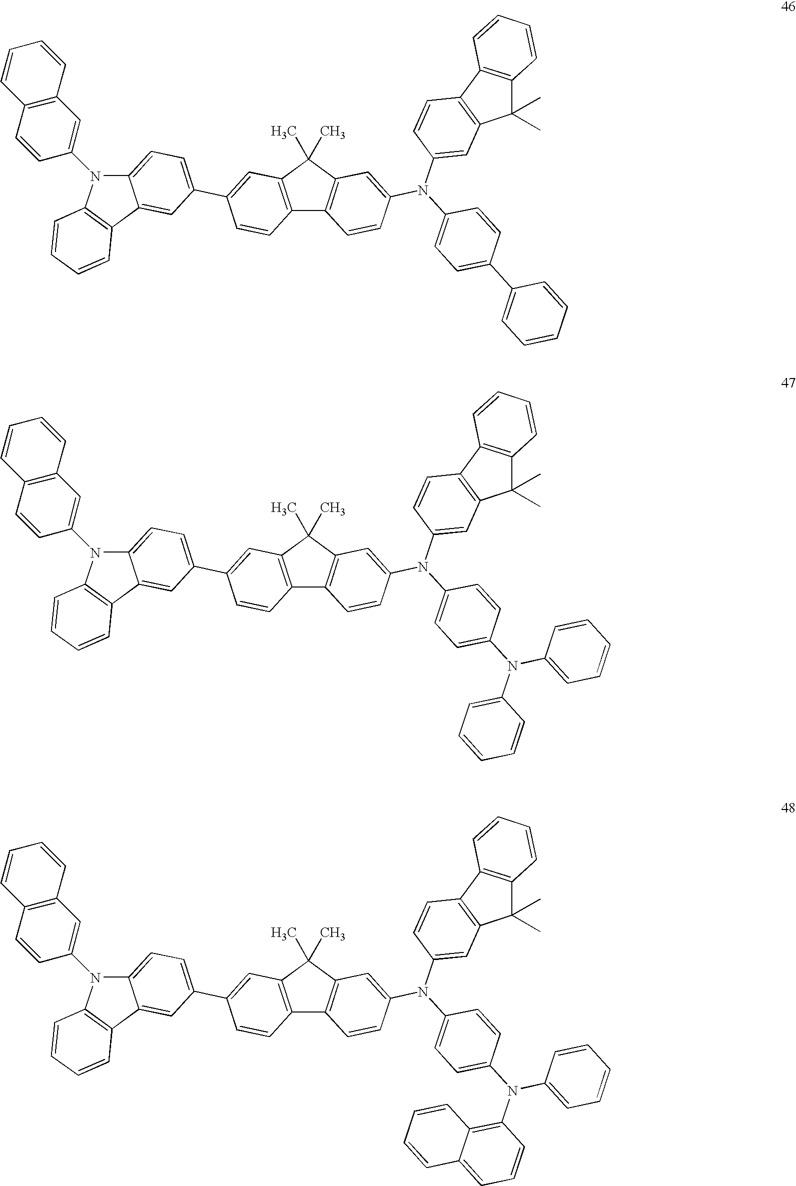

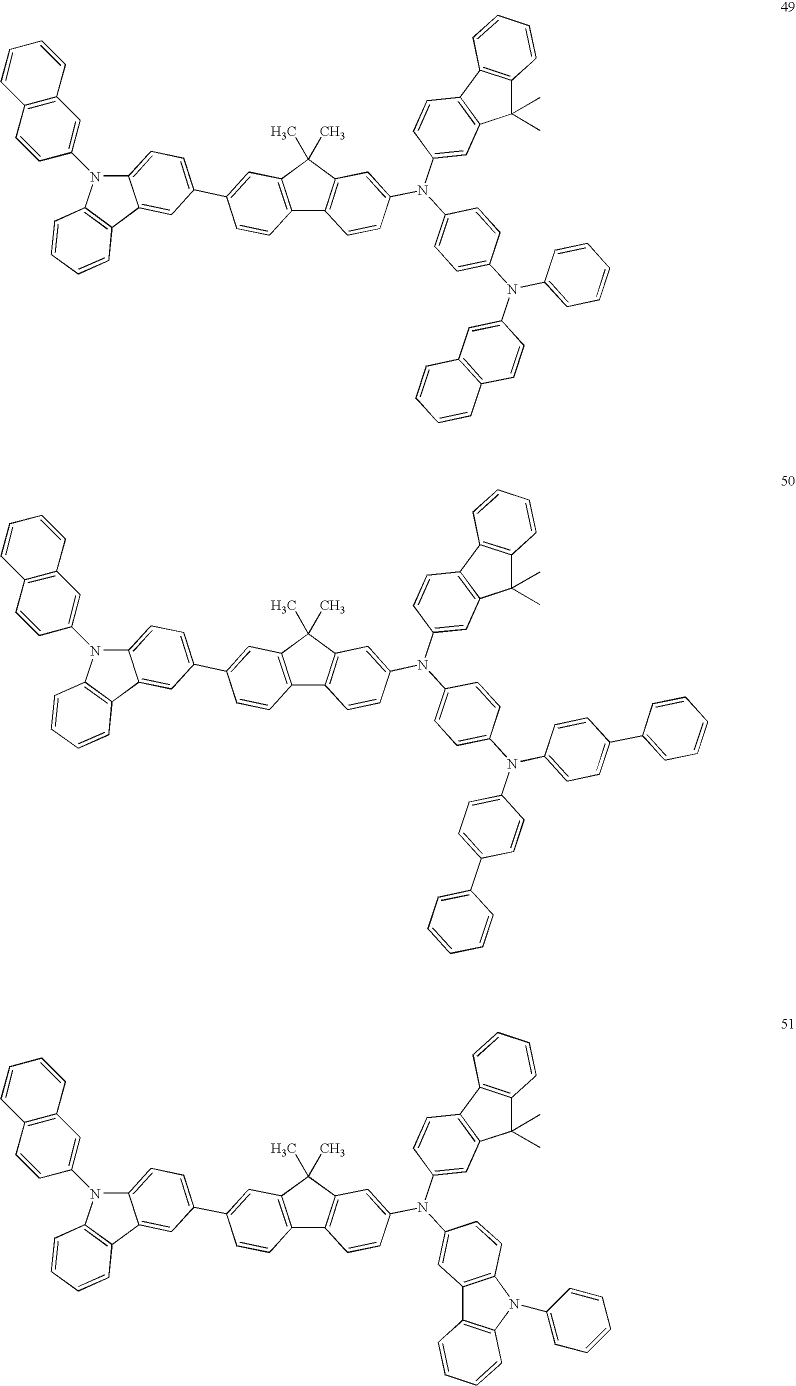

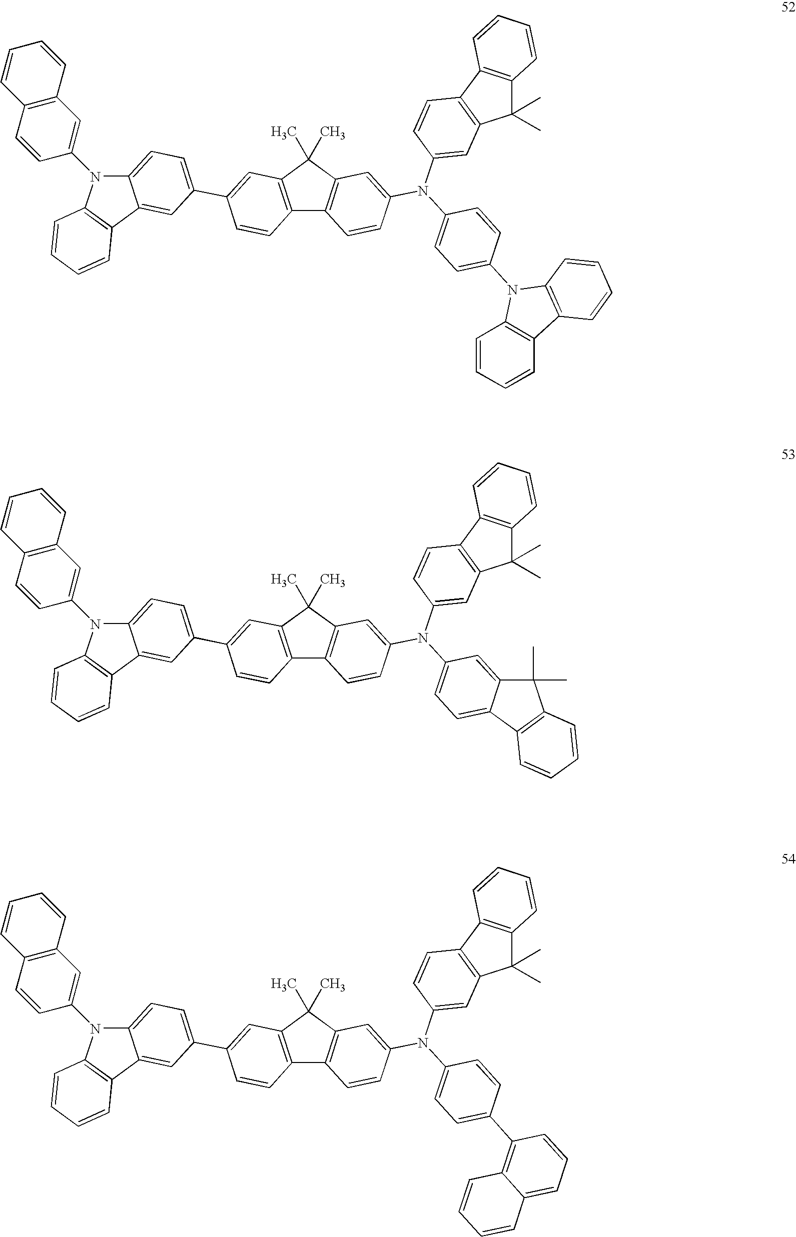

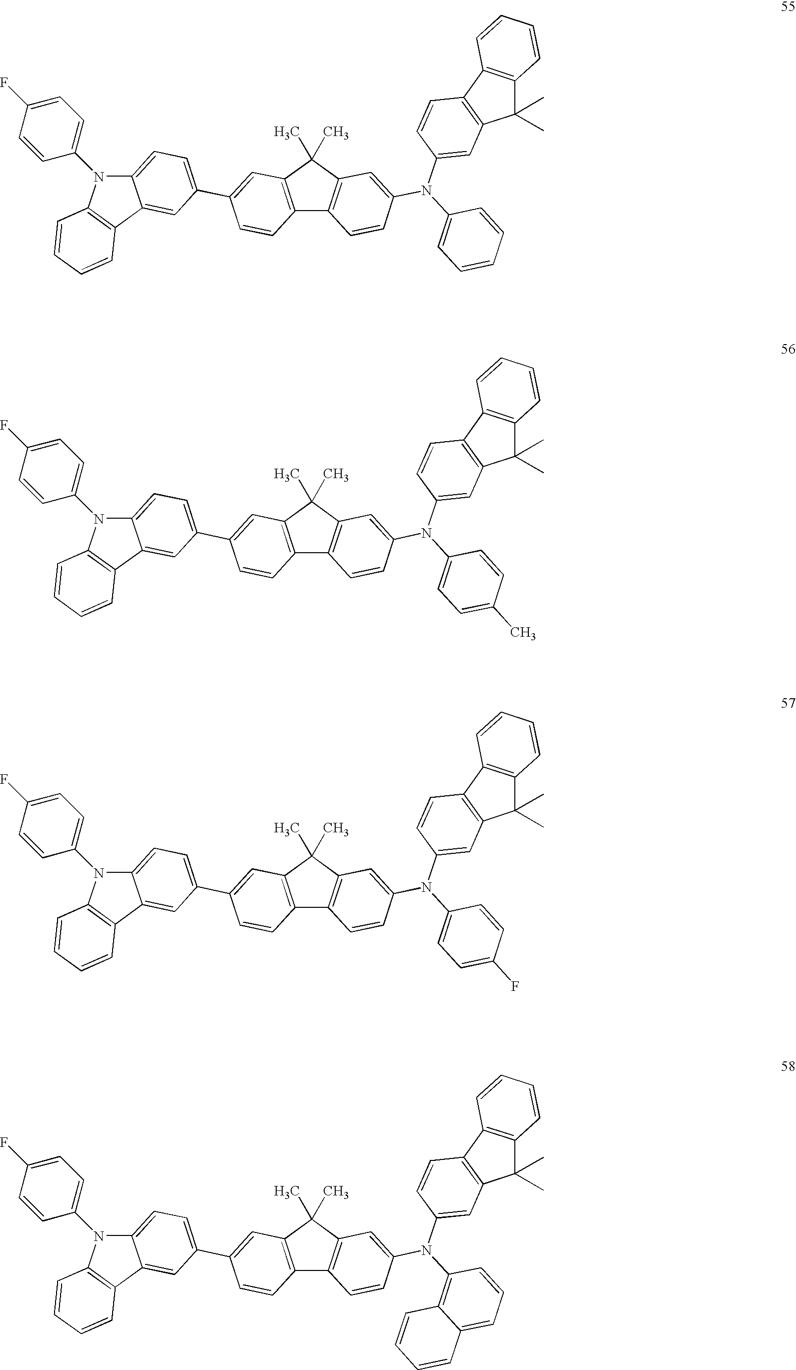

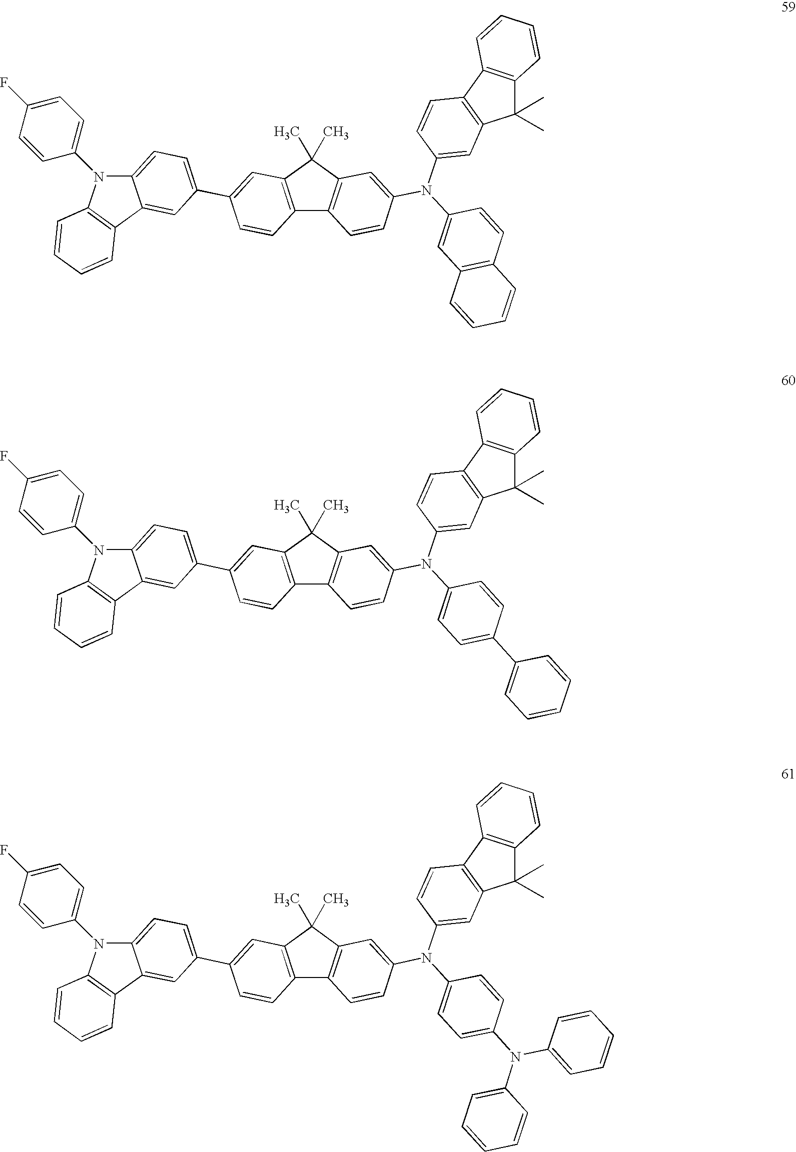

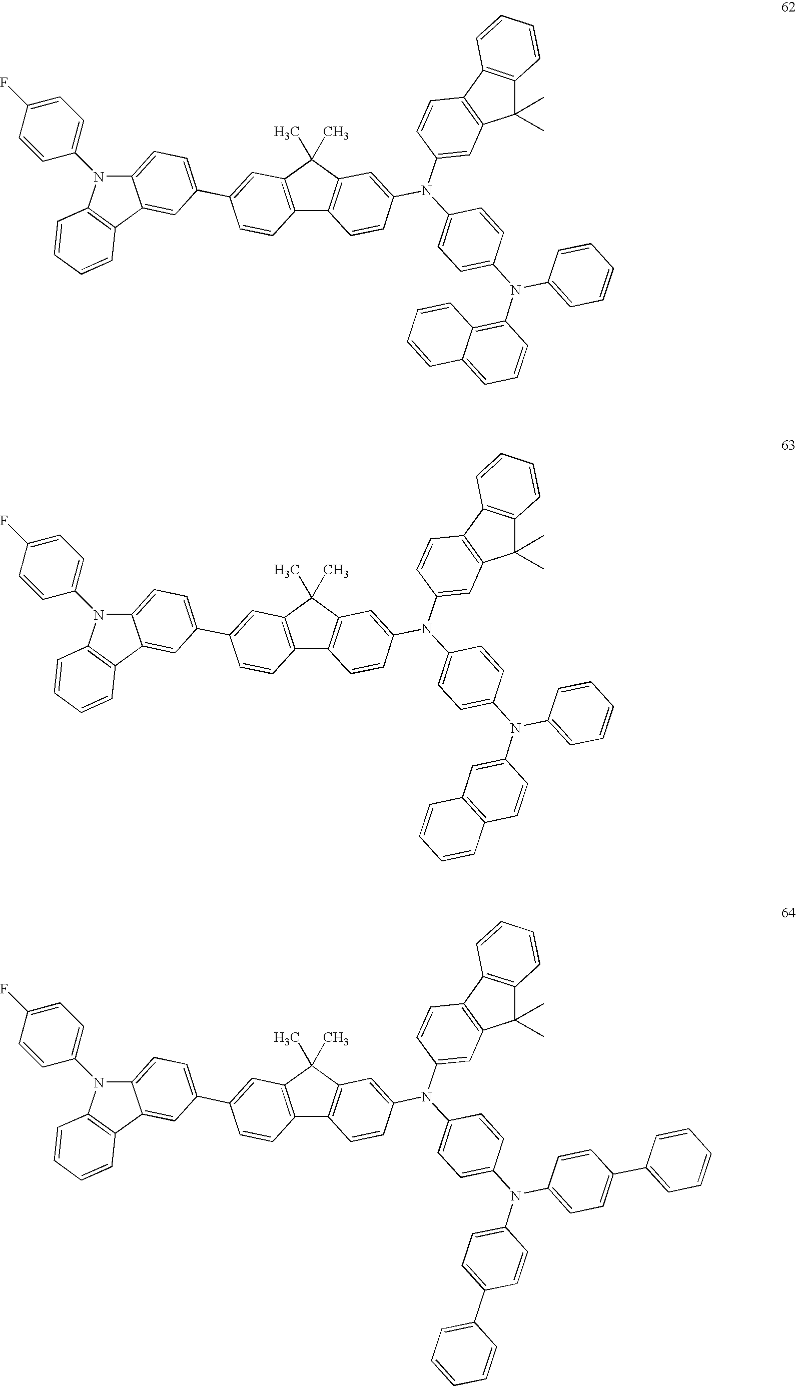

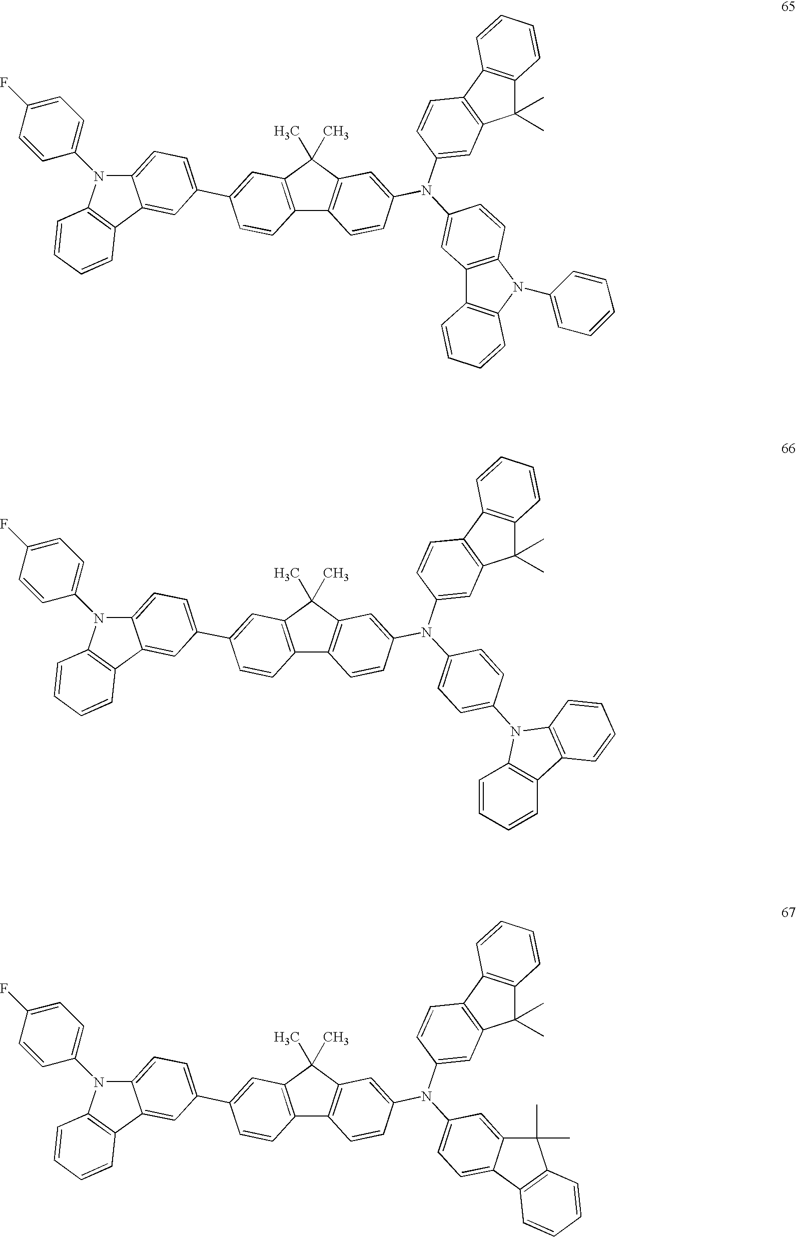

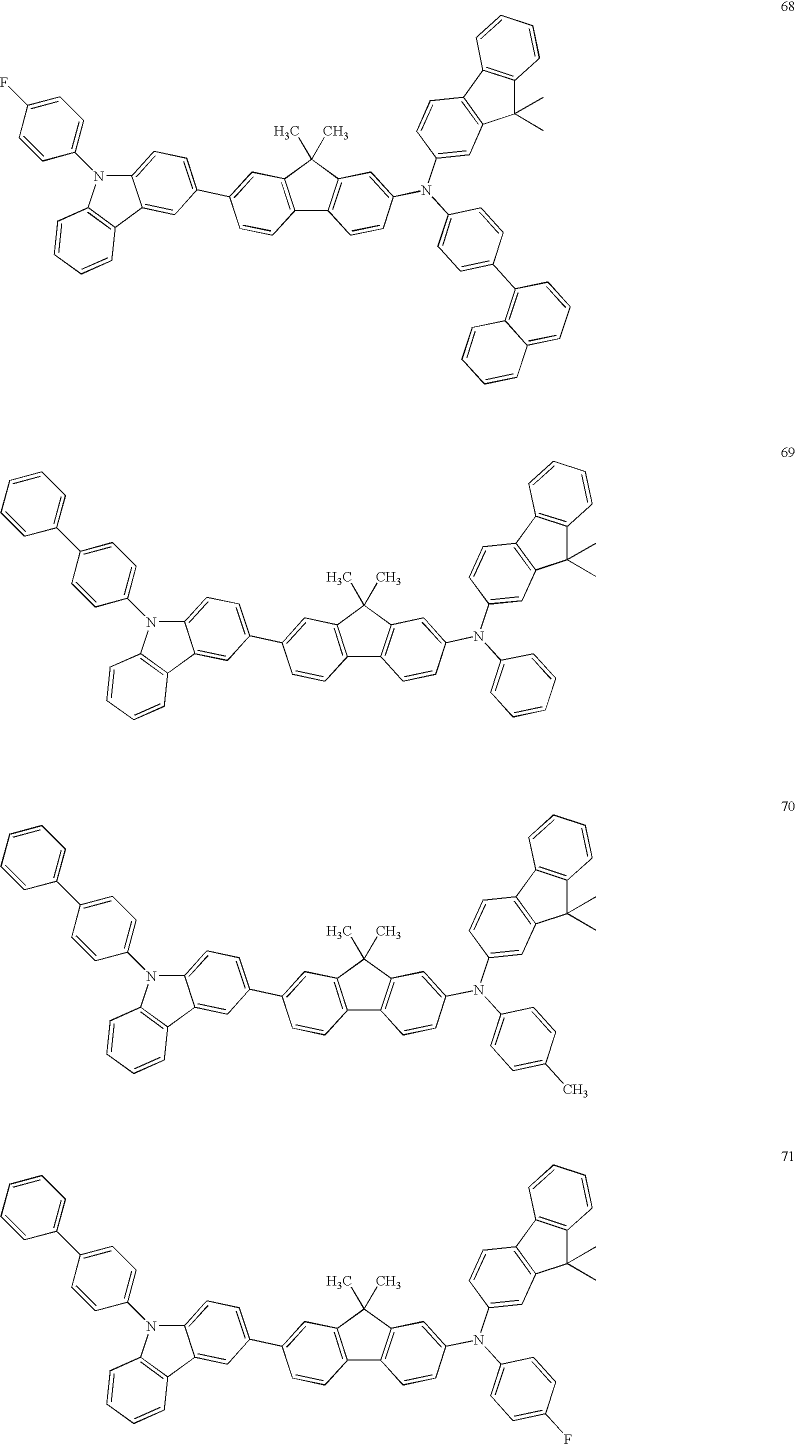

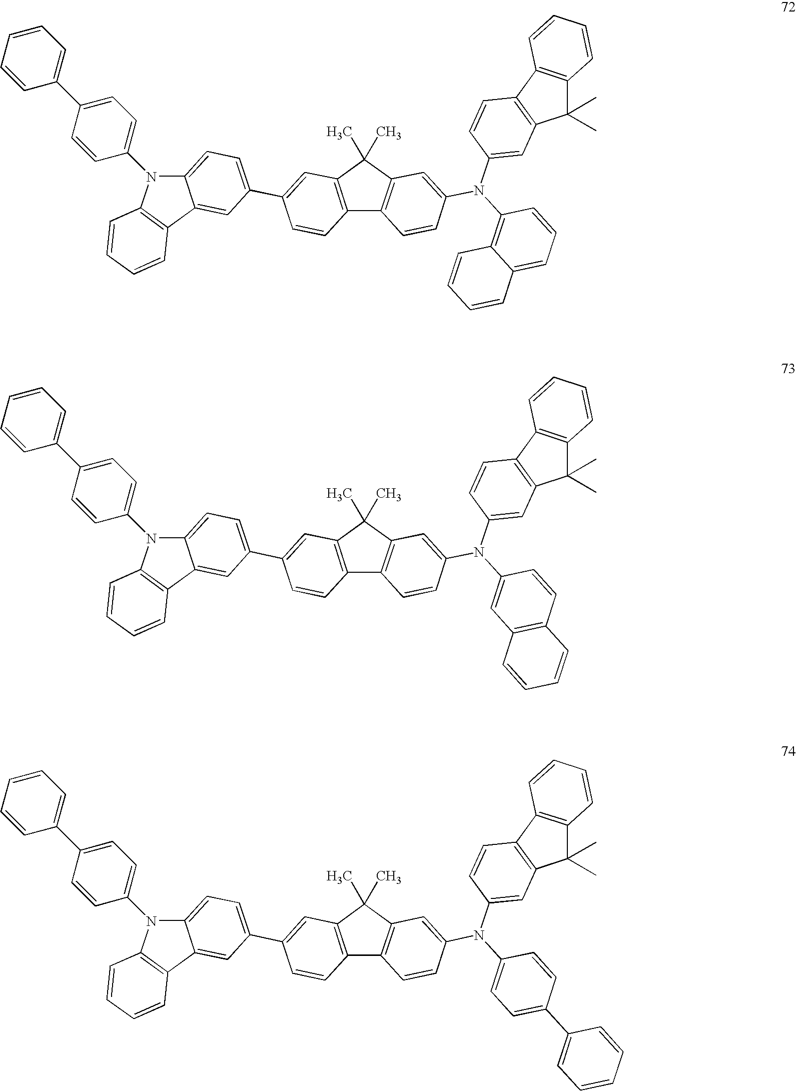

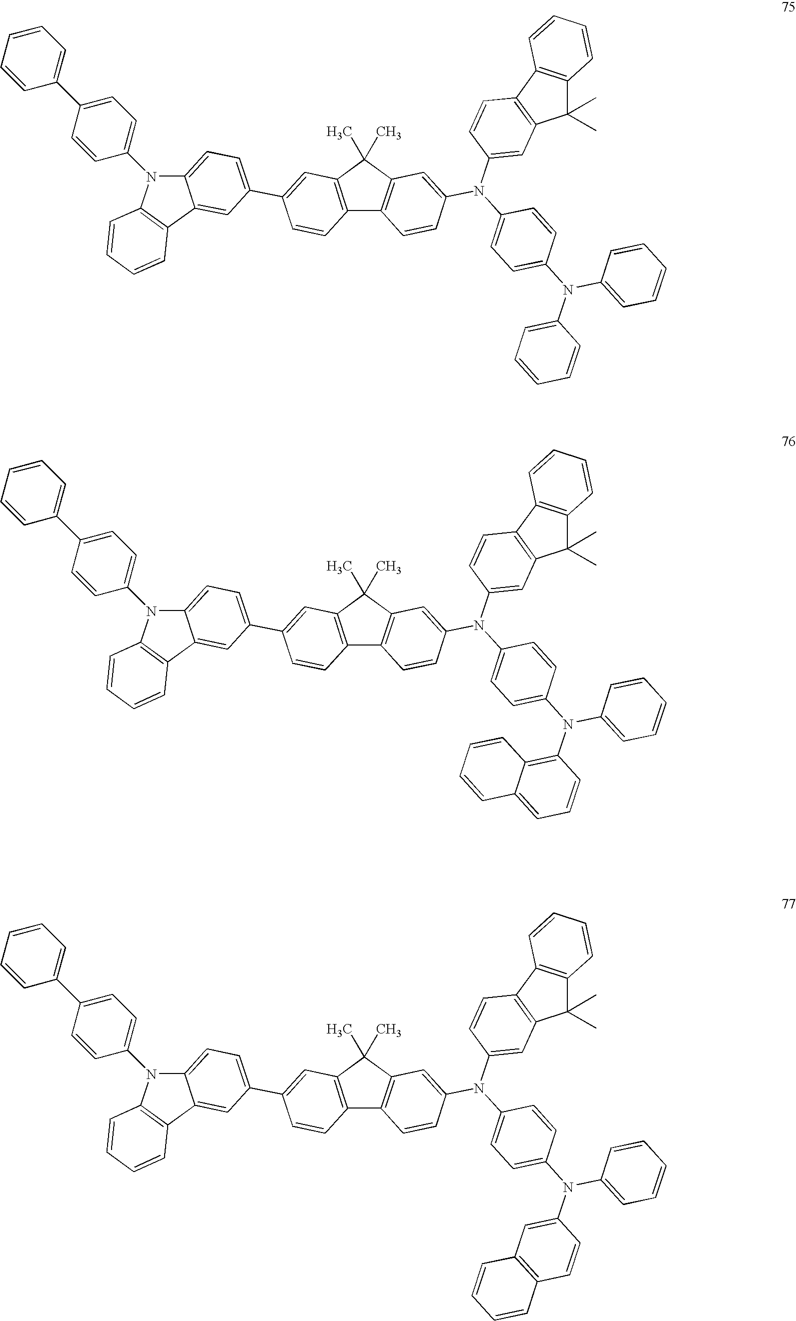

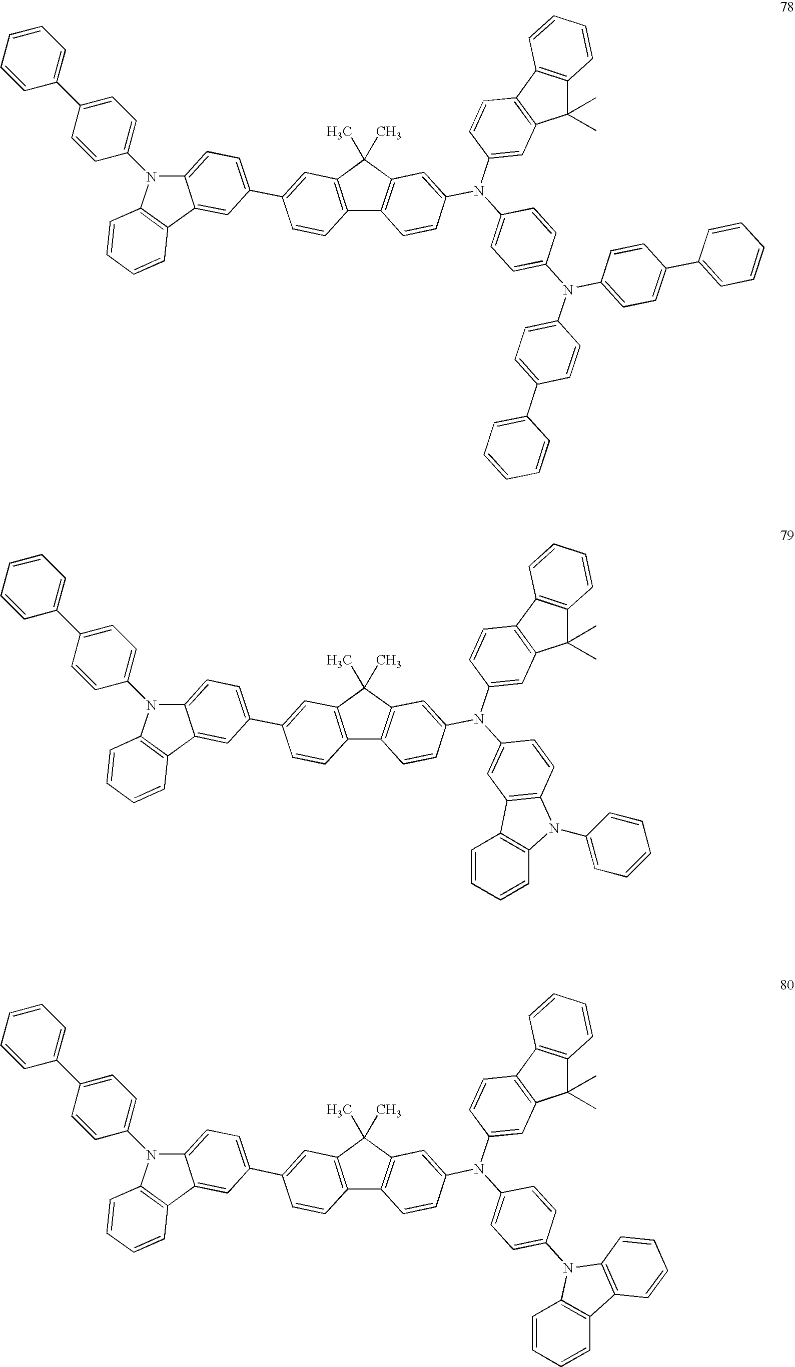

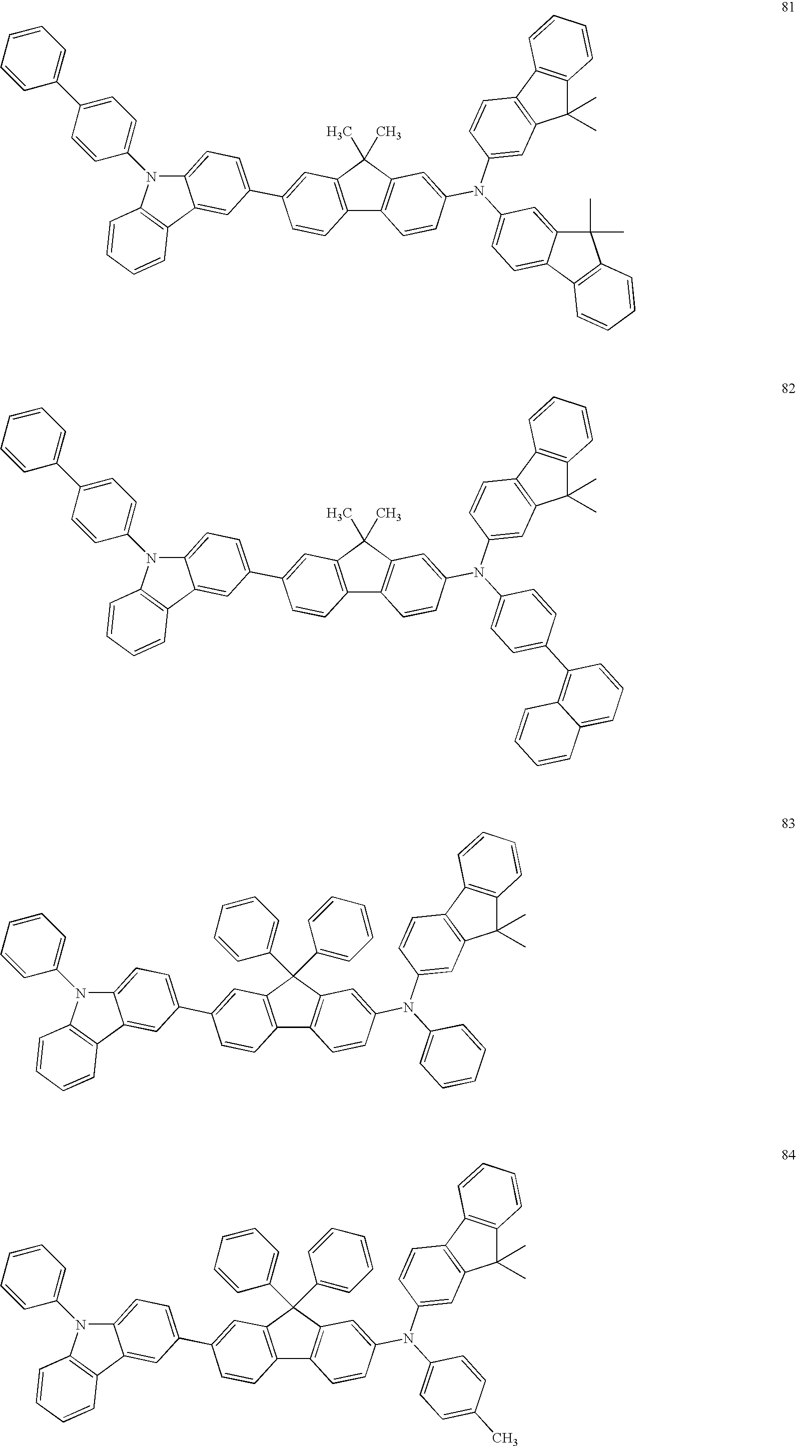

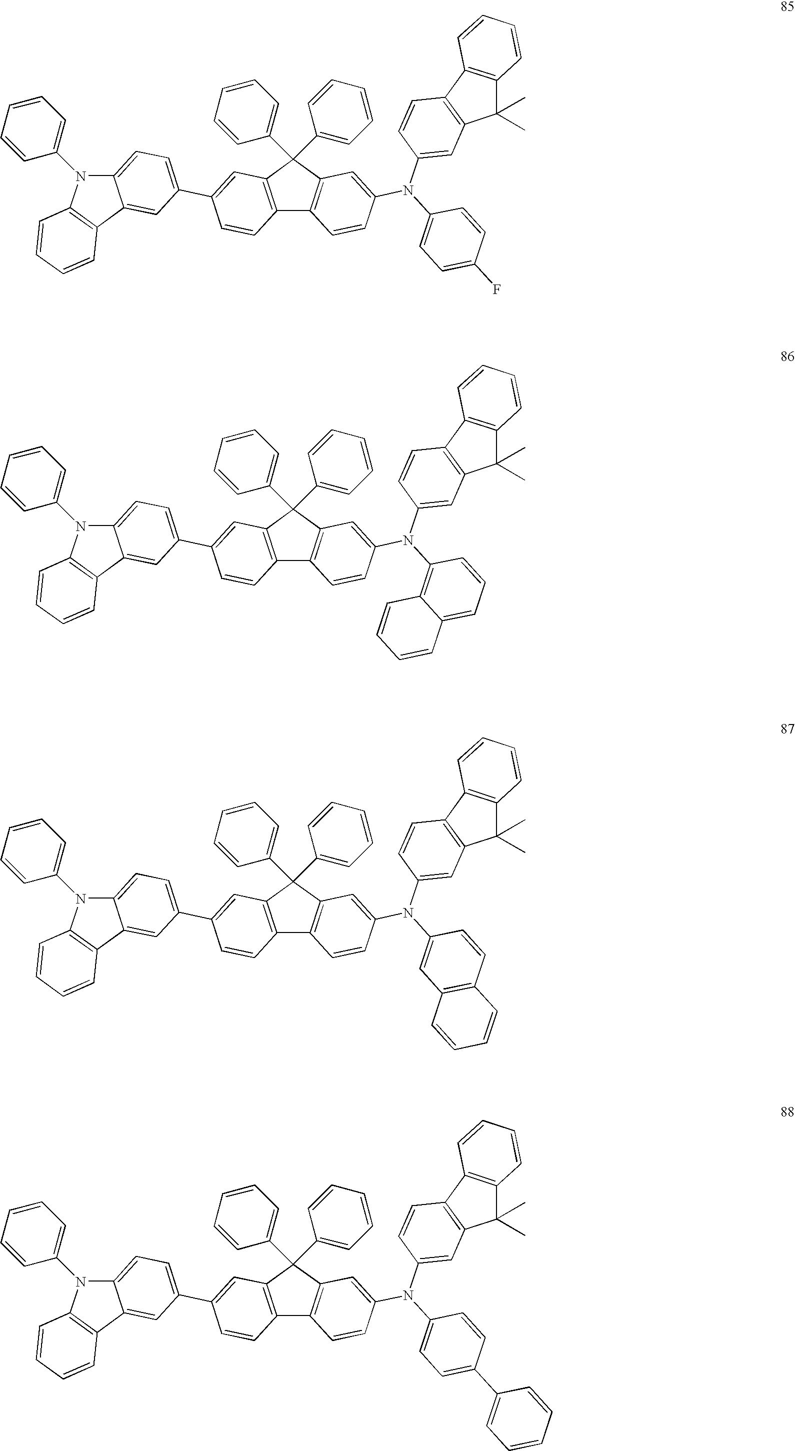

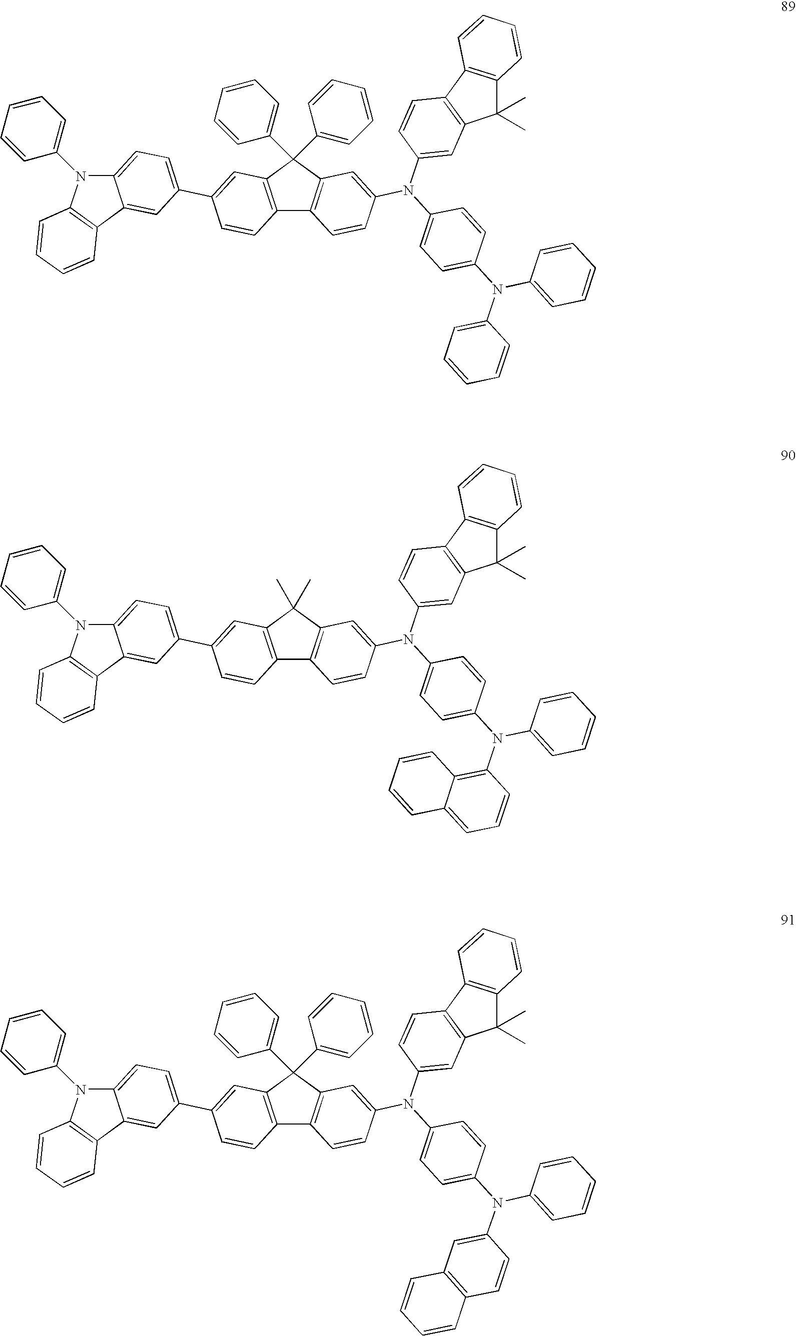

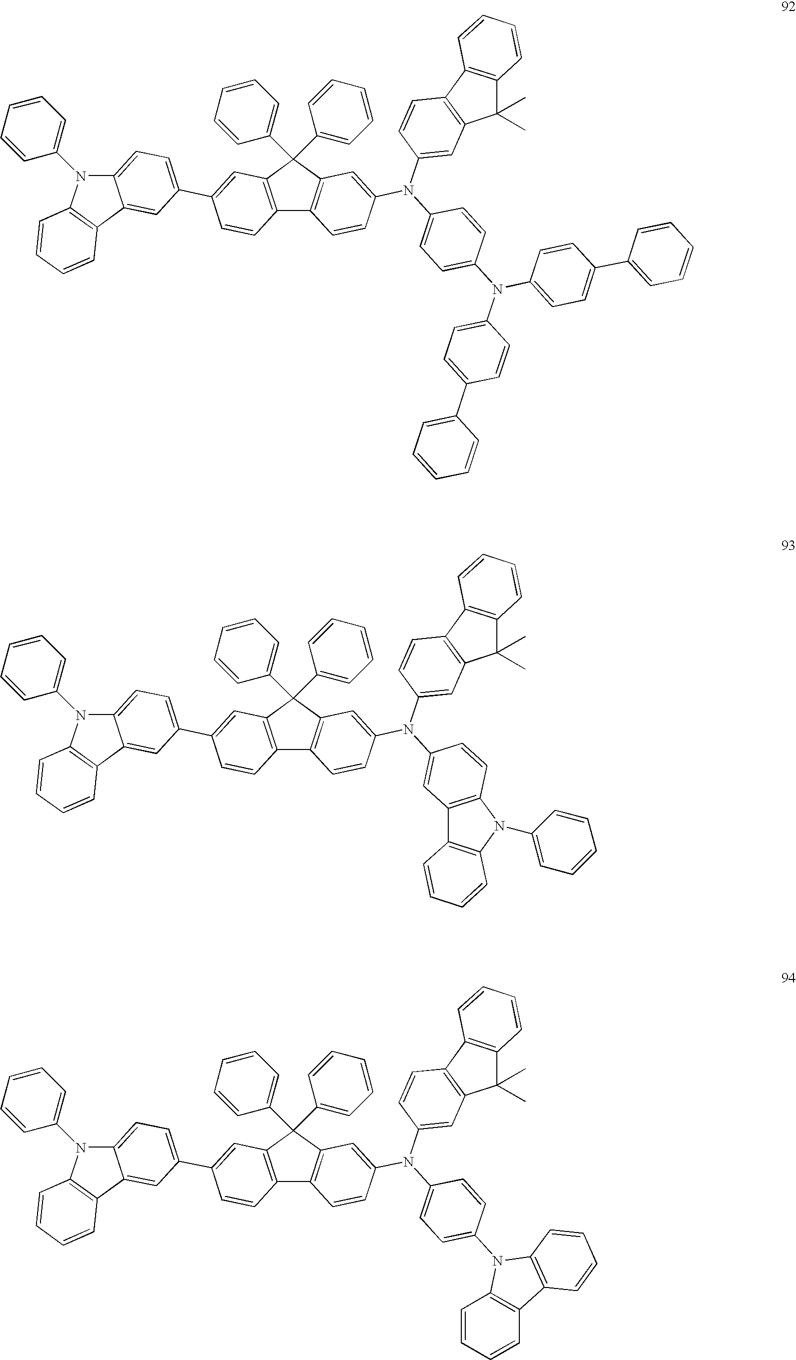

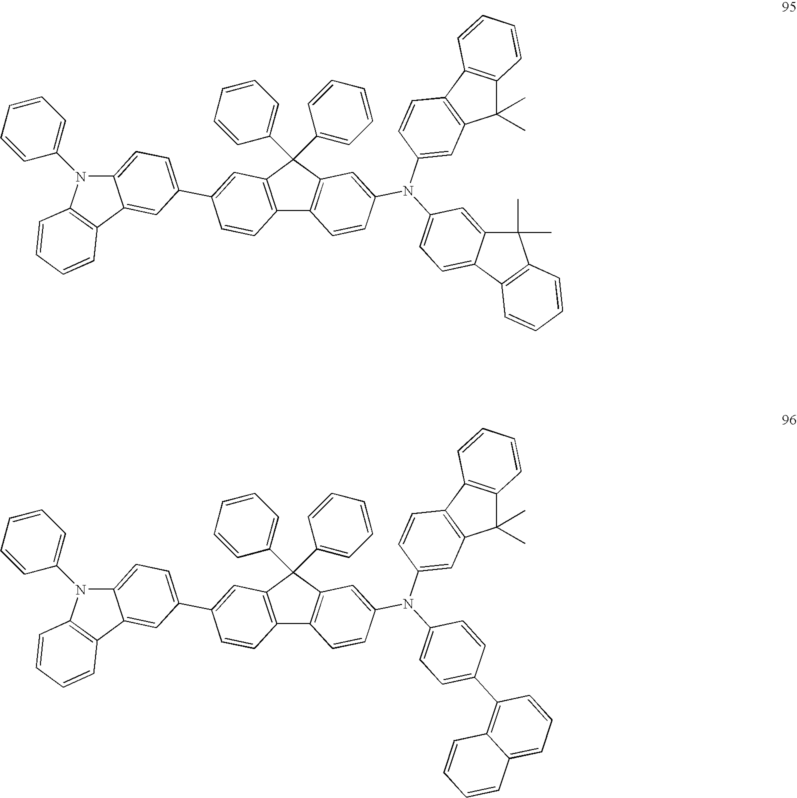

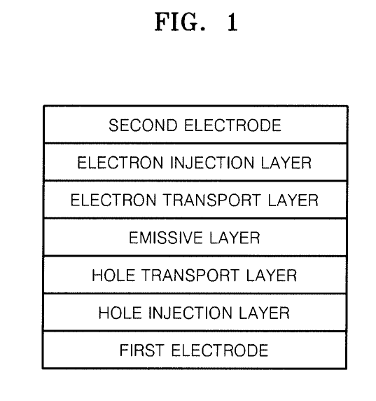

Referring now to FIG. 1, an organic light emitting device of an embodiment of the present invention includes a first electrode, a second electrode, and an organic film including the amine-based compound represented by Formula 1 between the first electrode and the second electrode. The organic film including the amine-based compound of Formula 1 may be a hole injection layer or a hole transport layer, and may also be a single film having both hole injection and hole transport capabilities. Alternatively, the organic film including the amine-based compound of Formula 1 may be an emissive layer. The amine-based compound of Formula 1 may also be used as a host material for phosphorescent or fluorescent materials of blue, green, or red colors.

In one embodiment, the organic film including the amine-based compound represented by Formula 1 may be a hole transport layer.

The first electrode may be an anode, and the second electrode may be a cathode, or vice versa.

The organic light emitting device may further include a layer selected from hole injection layers, hole transport layers, electron blocking layers, emissive layers, hole blocking layers, electron transport layers, electron injection layers, and combinations thereof. In one embodiment, the organic light emitting device includes an organic layer having two layers listed above.

The organic light emitting device according to an embodiment of the present invention may have a first electrode/hole injection layer/emissive layer/second electrode structure, a first electrode/hole injection layer/hole transport layer/emissive layer/electron transport layer/second electrode structure, or a first electrode/hole injection layer/hole transport layer/emissive layer/electron transport layer/electron injection layer/second electrode structure. Alternatively, the organic light emitting device may have a first electrode/single film with both hole injection and hole transport capabilities/emissive layer/electron transport layer/second electrode structure, or a first electrode/single film with both hole injection and hole transport capabilities/emissive layer/electron transport layer/electron injection layer/second electrode structure.

The organic light emitting device according to various embodiments of the present invention may be used in top emission type organic light emitting devices or bottom emission type organic light emitting devices.

A method of preparing the organic light-emitting device according to an embodiment of the present invention will now be described with reference to FIG. 1. The organic light-emitting device of FIG. 1 includes a substrate, a first electrode (anode), a hole injection layer, a hole transport layer, an emissive layer, an electron transport layer, an electron injection layer, and a second electrode (cathode).

First, the first electrode is formed by depositing a first electrode-forming material having a high work function on a substrate, by deposition or sputtering. The first electrode may either be an anode or a cathode. The substrate may be any substrate material conventionally used in the art. In one embodiment, the substrate is a glass substrate or a transparent plastic substrate, which has excellent mechanical strength, thermal stability, transparency, surface planarity, handling convenience, and water resistance. Nonlimiting examples of suitable first electrode-forming materials include indium tin oxide (ITO), indium zinc oxide (IZO), tin oxide (SnO₂), zinc oxide (ZnO), Al, Ag, Mg, and combinations thereof; all of which have excellent conductivity, and may be used to form a transparent or reflective electrode.

Next, the hole injection layer (HIL) may be formed on the first electrode, for example, by vacuum deposition, spin coating, casting, or Langmuir-Blodget (LB) deposition.

When the HIL is formed by vacuum deposition, the deposition conditions may vary according to the compound used, and the structure and thermal properties needed for different purposes. Generally, the deposition conditions are conducted at a deposition temperature ranging from 100 to 500° C., a vacuum pressure ranging from 10⁻⁸ to 10⁻³ torr, and at a deposition rate ranging from 0.01 to 100 Å/sec.

When the HIL is formed by spin coating, the coating conditions may vary according to the compound used, and the structure and thermal properties of the HIL needed for different purposes. Generally, the coating conditions are conducted at a coating speed ranging from about 2000 to about 5000 rpm, and a heat-treatment temperature for removing a solvent after coating ranging from 80 to 200° C.

The HIL material may be the amine-based compound represented by Formula 1 as previously described. Alternatively, other HIL materials known in the art may also be used. Nonlimiting examples of suitable HIL materials include phthalocyanine compounds such as copper phthalocyanine, 4,4′,4″-tris (3-methylphenylphenylamino) triphenylamine(m-MTDATA), N,N′-di(1-naphthyl)-N,N′-diphenylbenzidine) (NPB), TDATA, 2-TNATA, polyaniline/dodecylbenzenesulfonic acid (Pani/DBSA), poly(3,4-ethylenedioxythiophene)/poly(4-styrenesulfonate (PEDOT/PSS), polyaniline/camphor sulfonicacid (Pani/CSA), and PANI/PSS (polyaniline)/poly(4-styrenesulfonate).

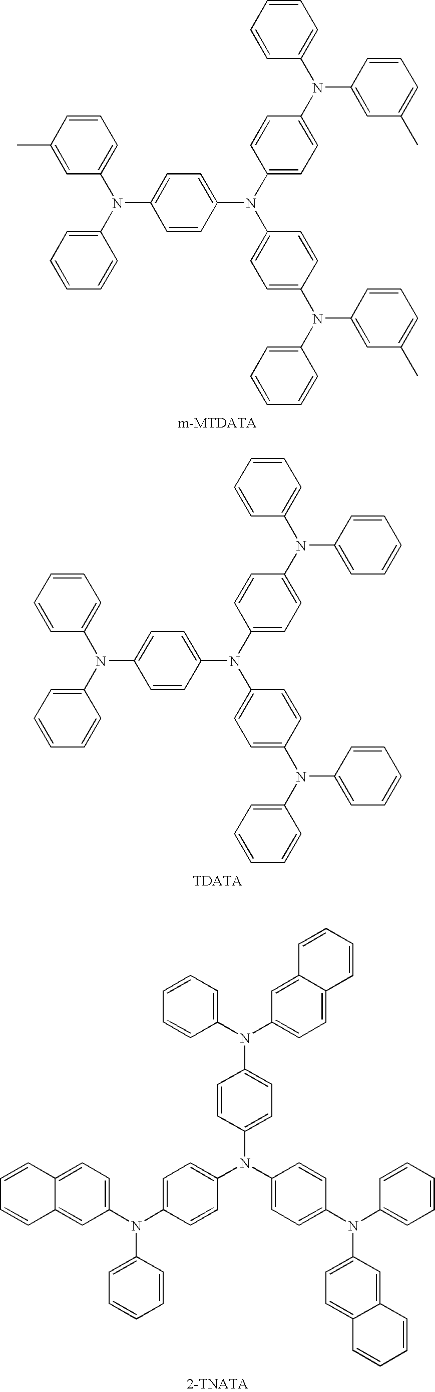

The thickness of the HIL may range from about 100 to 10000 Å, or more specifically, from 100 to 1000 Å. If the thickness of the HIL is within the above range, the HIL can have excellent hole injection properties without an increase in driving voltage.

Next, the hole transport layer (HTL) may be formed on the HIL, for example, by vacuum deposition, spin coating, casting, or LB deposition. When the HTL is formed by vacuum deposition or spin coating, the deposition or coating conditions may vary according to the compounds used. Generally, the conditions for forming the HTL may be similar to those for forming the HIL.

The HTL material may be the amine-based compound represented by Formula 1 as previously described. Alternatively, other HTL materials known in the art may be used. Nonlimiting examples of suitable HTL materials include carbazol derivatives such as N-phenylcarbazol, polyvinylcarbazol, and amine derivatives having aromatic condensed cycles such as NPB, N,N′-bis(3-methylphenyl)-N,N′-diphenyl-[1,1-biphenyl]-4,4′-diamine (TPD), and N,N′-di(naphthalene-1-yl) —N,N′-diphenyl benzidine (α-NPD).

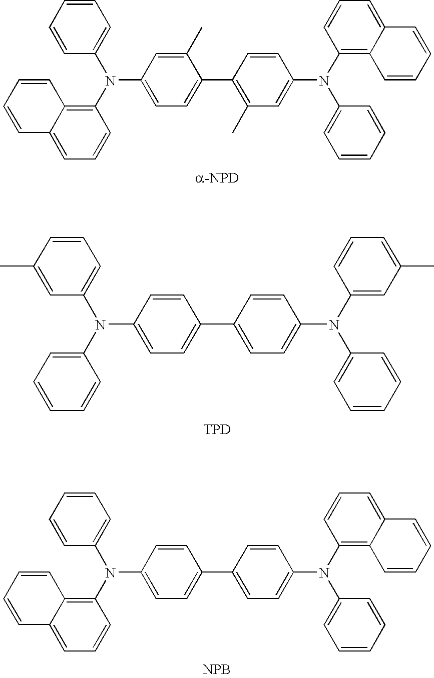

The thickness of the HTL may range from about 50 to about 1000 Å, or more specifically, from 100 to 600 Å. If the thickness of the HTL is within the above range, the HTL can have excellent hole transport properties without a substantial increase in driving voltage.

Next, the emissive layer (EML) may be formed on the HTL, for example, by vacuum deposition, spin coating, casting, or LB deposition. When the EML is formed by vacuum deposition or spin coating, the deposition or coating conditions may vary according to the compounds used, but the conditions may be similar to the conditions used to form the HIL.

The EML may include the amine-based compound represented by Formula 1 as previously described. In particular, the amine-based compound represented by Formula 1 may be used as a host or a dopant. The EML material can be any light-emitting materials, including host and dopant materials known in the art. The dopant may be either a fluorescent or a phosphorescent dopant material well known to those of ordinary skill in the art.

Nonlimiting examples of suitable host materials include Alq₃, CPB (4,4′-N,N′-dicarbazole-biphenyl), 9,10-di(naphthalene-2-yl)anthracene (ADN), and DSA (distyrylarylene).

Nonlimiting examples of suitable red dopant materials include PtOEP, Ir(piq)₃, Btp₂Ir(acac), and 4-(dicyanomethylene)-2-t-butyl-6-(1,1,7,7-tetramethyljulolidyl-9-enyl)-4H-pyran (DCJTB).

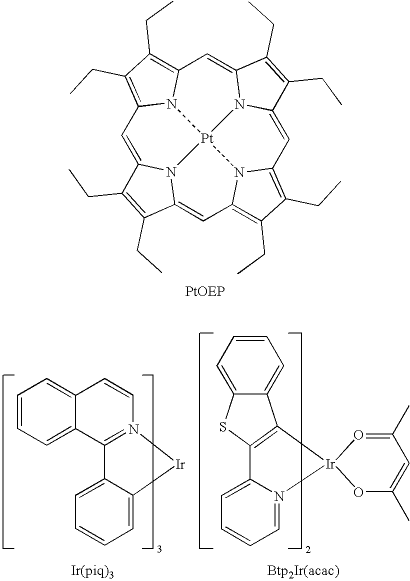

Nonlimiting examples of suitable green dopant materials include Ir(ppy)₃ (ppy=phenylpyridine), Ir(ppy)₂(acac), Ir(mpyp)₃, and C545T.

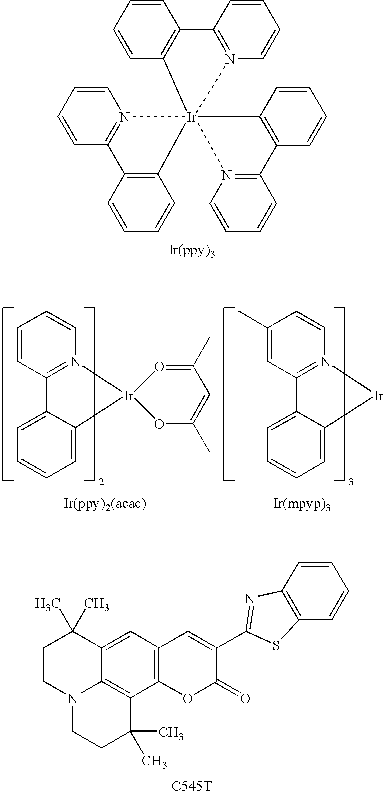

Nonlimiting examples of suitable blue dopant materials include F₂Irpic, (F₂ ppy)₂Ir(tmd), Ir(dfppz)₃, ter-fluorene, 4,4′-bis(4-diphenylaminostyryl)biphenyl (DPAVBi), and 2,5,8,11-tetra-tert-butylperylene (TBP).

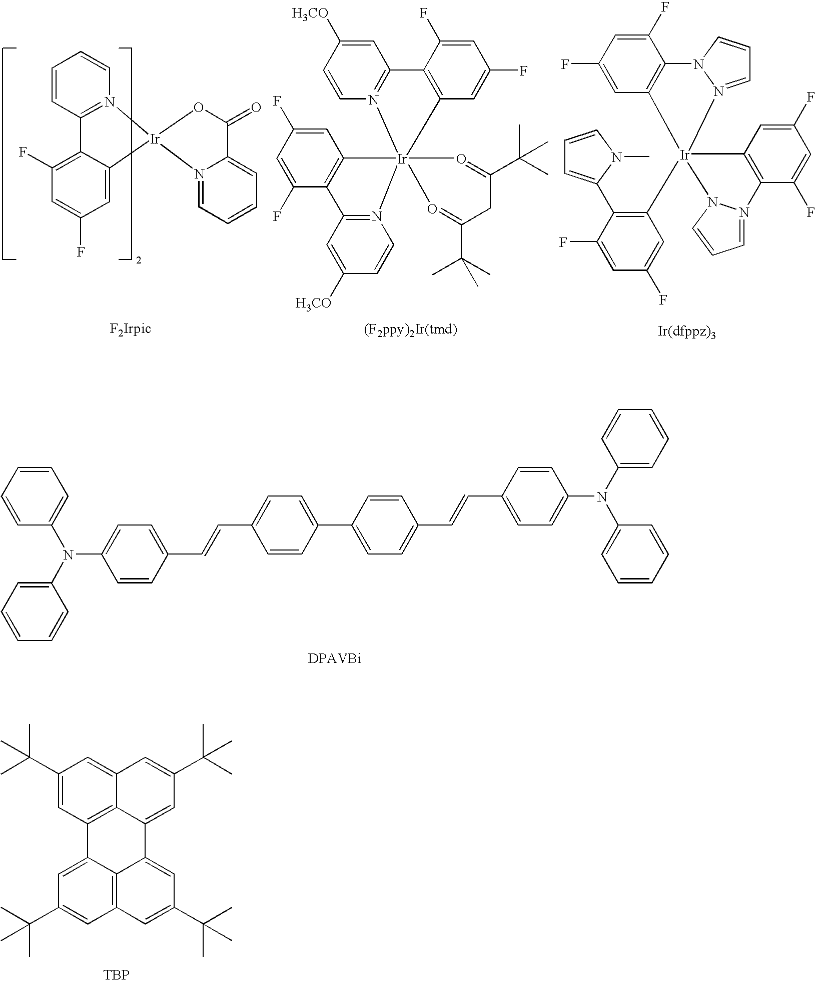

The content of the dopant may range from 0.1 to 20 parts by weight, or more specifically, from 0.5 to 12 parts by weight based on 100 parts by weight of the EML-forming material. That is, 100 parts by weight of the host and the dopant. When the content of the dopant is within the above range, concentration quenching may be substantially prevented.

The thickness of the EML may range from 100 to 1000 Å, or more specifically, from 200 to 600 Å. If the thickness of the EML is within the above range, the EML can have excellent light-emitting properties without a substantial increase in driving voltage.

If the EML includes a phosphorescent dopant, a hole blocking layer (HBL) may be formed on the EML to prevent triplet excitons or holes from diffusing to the electron transport layer. The HBL material can be any HBL materials known in the art. Nonlimiting examples of suitable HBL materials include oxadiazole derivatives, triazole derivatives, phenanthroline derivatives, Balq, and BCP.

The thickness of the HBL range from 50 to 1000 Å, or more specifically, from 100 to 300 Å. If the thickness of the HBL is less than 50 Å, the hole-blocking properties may deteriorate, and if the thickness of the HBL is greater than 1000 Å, the driving voltage may increase.

Next, the electron transport layer (ETL) may be formed on the HBL or EML, for example, by vacuum deposition, spin coating, or casting. When the ETL is formed by vacuum deposition or spin coating, the deposition or coating conditions may vary according to the compounds used. Generally, the process conditions for forming the ETL may be similar to those for forming the HIL.

The material used for the ETL can be any ETL-forming materials known in the art. Nonlimiting examples of suitable ETL materials include quinoline derivatives, tris(8-quinolinolate)aluminum (Alq₃), TAZ, and Balq.

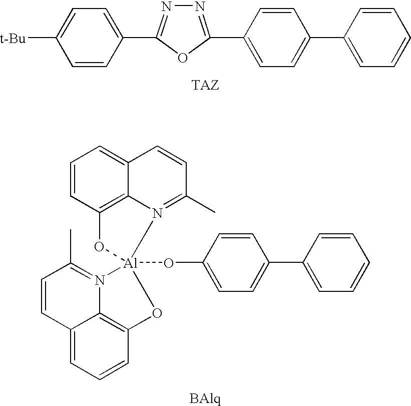

The thickness of the ETL may range from 100 to 1000 Å, or more specifically, from 100 to 500 Å. If the thickness of the ETL is within the above range, the ETL may have excellent electron transport properties without a substantial increase in driving voltage.

In addition, an electron injection layer (EIL), which has a function of facilitating the injection of electrons from the cathode may be formed on the ETL.

Nonlimiting examples of suitable EIL materials include LiF, NaCl, CsF, Li₂O, and BaO. The deposition or coating conditions may vary according to the compounds used, but may be similar to the conditions that are used to form the HIL.

The thickness of the EIL may range from 1 to 100 Å, or more specifically, from 5 to 90 Å. If the thickness of the EIL is within the above range, the EIL may have excellent electron injection properties without a substantial increase in driving voltage.

Finally, the second electrode may be formed on the EIL by vacuum deposition or sputtering. The second electrode may be used as a cathode or an anode. The material for forming the second electrode may be a metal, an alloy, or an electrically conductive compound with a low work function. Nonlimiting examples of such materials include Li, Mg, Al, Al—Li, Ca, Mg—In, and Mg—Ag. In addition, the second electrode may be formed of a transparent material such as ITO or IZO to provide a top-emission type organic light emitting device.

The organic light-emitting device according to various embodiments of the present invention may be used in various forms of flat-panel display devices, such as passive matrix organic light emitting display devices or active matrix organic light emitting display devices. When the organic light emitting device is included in an active matrix organic light emitting display device including a thin-film transistor, the first electrode on the substrate is a pixel electrode, which is electrically connected to a source electrode or a drain electrode of the thin-film transistor. The organic light emitting device may also be included in a flat panel display device having a double-sided screen.

The following Experimental Examples are provided for illustrative purposes only, and do not limit the scope of the present invention.

### EXAMPLES

### Synthesis Example 1

### Synthesis of Intermediate 1

2.433 g of phenylcarbazole (10 mmol) was added into 100 ml of 80% acetic acid, and 1.357 g of Iodine (I₂) (5.35 mmol) and 0.333 g of ortho-periodic acid (H₅IO₆) (1.46 mmol) in a solid state were added thereto to form a mixture. The mixture was stirred at 80° C. for 2 hours in a nitrogen atmosphere. A reaction was allowed to take place and terminated.

After the reaction was terminated, the mixture was extracted with 50 ml of ethylether three times. An organic layer was collected and dried using magnesium sulfate to evaporate the solvent. The residue was separately purified by silica gel column chromatography to obtain 3.23 g (yield 87%) of white solid of Intermediate 1. Intermediate 1 was evaluated by NMR, and the result thereof is shown below.

¹H NMR (CDCl₃, 300 MHz) δ (ppm) 8.43 (d, 1H), 8.05 (d, 1H), 7.62 (dd, 1H), 7.61-7.75 (m, 2H), 7.51-7.43 (m, 3H), 7.41-7.35 (m, 2H), 7.27 (dd, 1H), 7.14 (d, 1H)

### Synthesis Example 2

### Synthesis of Intermediate 2

Intermediate 2 was synthesized to obtain a yield of 85% in the same manner as in the synthesis of Intermediate 1, except that naphthylcarbazole was used instead of phenylcarbazole. Intermediate 2 was evaluated by NMR, and the result thereof is shown below.

¹H NMR (CDCl₃, 300 MHz) δ (ppm) 8.29 (s, 1H), 8.13 (d, 1H), 7.92 (d, 1H), 7.80-7.73 (m, 3H), 7.59-7.49 (m, 5H), 7.37-7.29 (m, 3H)

### Synthesis Example 3

### Synthesis of Intermediate 3

Intermediate 3 was synthesized to obtain a yield of 83% in the same manner as in the synthesis of Intermediate 1, except that 4-fluorophenylcarbazole was used instead of phenylcarbazole. Intermediate 3 was evaluated by NMR, and the result thereof is shown below.

¹H NMR (CDCl₃, 300 MHz) δ (ppm) 8.30 (s, 1H), 8.13-8.11 (m, 1H), 7.74 (d, 1H), 7.56-7.52 (m, 3H), 7.37-7.29 (m, 3H), 7.08-7.03 (m, 2H)

### Synthesis Example 4

### Synthesis of Intermediate 4

Intermediate 4 was synthesized to obtain a yield of 62% in the same manner as in the synthesis of Intermediate 1, except that biphenylcarbazole was used instead of phenylcarbazole. Intermediate 4 was evaluated by NMR, and the result thereof is shown below.

¹H NMR (CDCl₃, 300 MHz) δ (ppm) 8.30 (s, 1H), 8.13-8.11 (m, 1H), 7.74 (d, 1H), 7.59-7.53 (m, 3H), 7.40-7.28 (m, 8H), 6.64-6.60 (m, 2H)

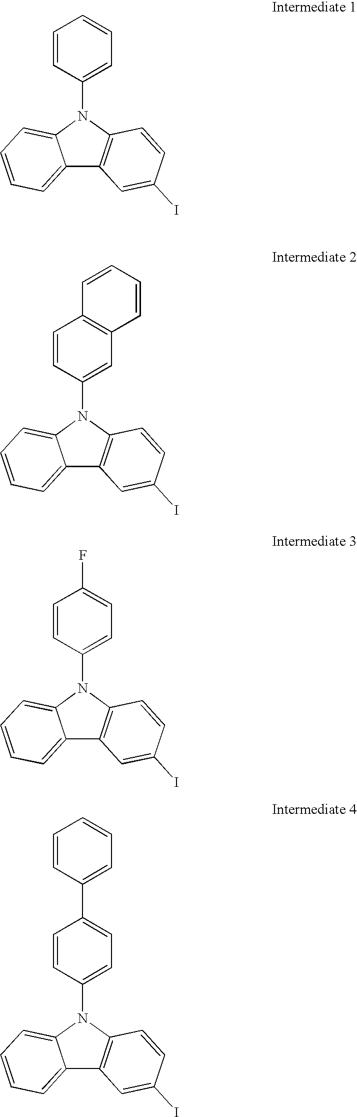

### Synthesis Example 5

### Synthesis of Intermediate 5

17.6 g of 2,7-dibromo-9,9-dimethylfluorene (50 mmol) was dissolved in 150 ml of diethyl ether, and a normal butyl lithium (20 ml, 2.5 M in hexane) solution maintained at −78° C. was added thereto. The temperature was raised slowly to room temperature after 30 minutes. After another 30 minutes, a solution of 23 ml (100 mmol) triisopropyl borate dissolved in 50 ml of diethyl ether maintained at −78° C. was slowly added thereto to form a mixture. The mixture was stirred for 5 hours at room temperature, and water was added thereto, and the resulting mixture was washed three times with diethyl ether (200 ml). The washed diethyl ether layer was dried with MgSO₄, and was further dried under reduced pressure to obtain a product, which was recrystallized with normal hexane to obtain 10.3 g (yield: 65%) of white solid Intermediate 5. Intermediate 5 was evaluated by NMR, and the result thereof is shown below.

¹H NMR (CDCl₃, 400 MHz) δ (ppm)-8.09 (s, 20H), 7.84-7.81 (dd, 1H), 7.75-7.72 (m, 2H), 7.67-7.65 (dd, 1H), 7.49-7.46 (dd, 1H), 7.40 (d, 1H), 1.85 (s, 6H)

### Synthesis Example 6

### Synthesis of Intermediate 6

Intermediate 6 was synthesized to obtain a yield of 58% in the same manner as in the synthesis of Intermediate 5, except that 2,7-dibromo-9,9-diphenylfluorene was used instead of 2,7-dibromo-9,9-dimethylfluorene. Intermediate 6 was evaluated by NMR, and the result thereof is shown below.

¹H NMR (CDCl₃, 400 MHz) δ (ppm)-8.27 (d, 1H), 8.09 (s, 20H), 7.94-7.92 (dd, 1H), 7.81 (d, 1H), 7.74 (d, 1H), 7.69 (d, 1H), 7.60-7.51 (m, 5H), 7.18 (t, 4H), 7.02-6.97 (m, 2H)

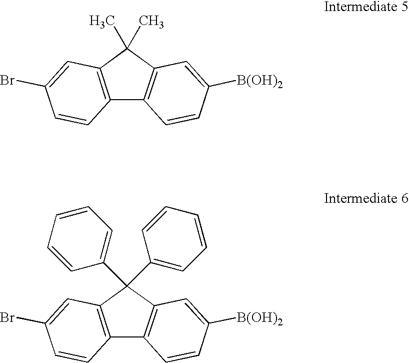

### Synthesis Example 7

### Synthesis of Intermediate 7

8.2 g (30 mmol) of 2-bromo-9,9-dimethylfluorene, 4.1 mL (45 mmol) of aniline, 4.3 g (45 mmol) of t-BuONa, 0.55 g (0.6 mmol) of Pd₂(dba)₃, and 0.12 g (0.6 mmol) of P(t-Bu)₃ were dissolved in 100 ml of toluene to form a mixture. The mixture was stirred at 90° C. for 3 hours. A reaction was allowed to take place and after the reaction was completed, the reaction mixture was cooled to room temperature. The mixture was extracted three times with 100 ml of distilled water and diethyl ether. An organic layer was collected and was dried using magnesium sulfate to evaporate the solvent. The residue was separately purified by silica gel column chromatography to obtain 7.87 g (yield 92%) of Intermediate 7. Intermediate 7 was evaluated by NMR, and the results thereof are shown below.

¹H NMR (CDCl₃, 400 MHz) δ (ppm)-7.82 (d, 1H), 7.54-7.49 (m, 2H), 7.27-7.21 (m, 3H), 7.12-7.08 (m, 3H), 6.97-6.93 (m, 1H), 6.90-6.86 (m, 1H), 6.59-6.56 (m, 1H), 5.44 (NH), 1.85 (s, 6H)

¹³C NMR (CDCl₃, 100 MHz) δ (ppm)-149.1, 146.8, 142.2, 140.0, 134.6, 133.5, 129.4, 127.8, 127.1, 126.8, 120.4, 119.7, 119.1, 117.4, 109.0, 107.5, 44.9, 24.5

### Synthesis Example 8

### Synthesis of Intermediate 8

Intermediate 8 was synthesized to obtain a yield of 78% in the same manner as in the synthesis of Intermediate 7, except that 4-fluoroaniline was used instead of aniline. Intermediate 8 was evaluated by NMR, and the results thereof are shown below.

¹H NMR (CDCl₃, 400 MHz) δ (ppm)-7.95-7.91 (m, 2H), 7.82 (d, 1H), 7.54-7.49 (m, 2H), 7.24-7.19 (m, 3H), 7.12 (d, 1H), 6.97-6.93 (m, 1H), 6.59-6.56 (dd, 1H), 5.44 (s, NH), 1.85 (s, 6H)

¹³C NMR (CDCl₃, 100 MHz) δ (ppm)-161.0, 154.5, 149.1, 146.8, 140.0, 138.3, 134.6, 132.5, 127.8, 127.1, 126.8, 120.4, 119.7, 116.5, 115.8, 112.3, 112.1, 109.6, 107.5, 44.9, 24.5

### Synthesis Example 9

### Synthesis of Intermediate 9

Intermediate 9 was synthesized to obtain a yield of 83% in the same manner as in the synthesis of Intermediate 7, except that 1-bromonaphthalene was used instead of aniline. Intermediate 9 was evaluated by NMR, and the results thereof are shown below.

¹H NMR (CDCl₃, 400 MHz) δ (ppm)-8.41 (dd, 1H), 7.82 (d, 1H), 7.72 (d, 1H), 7.59-7.36 (m, 5H), 7.24-7.09 (m, 4H), 6.97-6.93 (m, 1H), 6.55 (dd, 1H), 5.83 (s, NH), 1.85 (s, 6H)

¹³C NMR (CDCl₃, 100 MHz) δ (ppm)-149.1, 148.7, 144.3, 140.0, 137.2, 134.6, 133.3, 128.8, 128.2, 127.8, 127.1, 126.8, 125.0, 124.5, 122.4, 122.3, 121.3, 119.7, 116.8, 109.2, 107.7, 99.0, 44.9, 24.5

### Synthesis Example 10

### Synthesis of Intermediate 10

Intermediate 10 was synthesized to obtain a yield of 85% in the same manner as in the synthesis of Intermediate 7, except that 4-aminobiphenyl was used instead of aniline. Intermediate 10 was evaluated by NMR, and the results thereof are shown below.

¹H NMR (CDCl₃, 400 MHz) δ (ppm)-7.82 (d, 1H), 7.60-7.49 (m, 6H), 7.40-7.29 (m, 3H), 7.24-7.20 (m, 1H), 7.12 (d, 1H), 6.95 (t, 1H), 6.79-6.75 (m, 2H), 6.59-6.56 (m, 1H), 5.44 (s, NH), 1.85 (s, 6H)

¹³C NMR (CDCl₃, 100 MHz) δ (ppm)-149.1, 146.8, 140.0, 138.8, 137.4, 134.9, 134.6, 132.9, 129.9, 128.8, 127.8, 127.2, 127.1, 126.8, 120.4, 119.7, 116.3, 109.0, 107.5, 44.9, 24.5

### Synthesis Example 11

### Synthesis of Intermediate 11

Intermediate 11 was synthesized to obtain a yield of 74% in the same manner as in the synthesis of Intermediate 7, except that N,N′-dibiphenyl-1,4-phenylenediamine was used instead of aniline. Intermediate 11 was evaluated by NMR, and the results thereof are shown below.

¹H NMR (CDCl₃, 400 MHz) δ (ppm)-7.82 (d, 1H), 7.76-7.64 (m, 8H), 7.54-7.49 (m, 2H), 7.40-7.11 (m, 10H), 6.97-6.93 (m, 1H), 6.60-6.43 (m, 7H), 5.44 (s, NH), 1.85 (s, 6H)

¹³C NMR (CDCl₃, 100 MHz) δ (ppm)-149.1, 147.9, 146.8, 143.8, 140.0, 136.9, 136.4, 135.4, 134.6, 134.2, 132.3, 128.9, 128.8. 127.8, 127.2, 127.1, 126.8, 120.4, 119.7, 117.7, 114.6, 109.0, 107.5, 44.9, 24.5

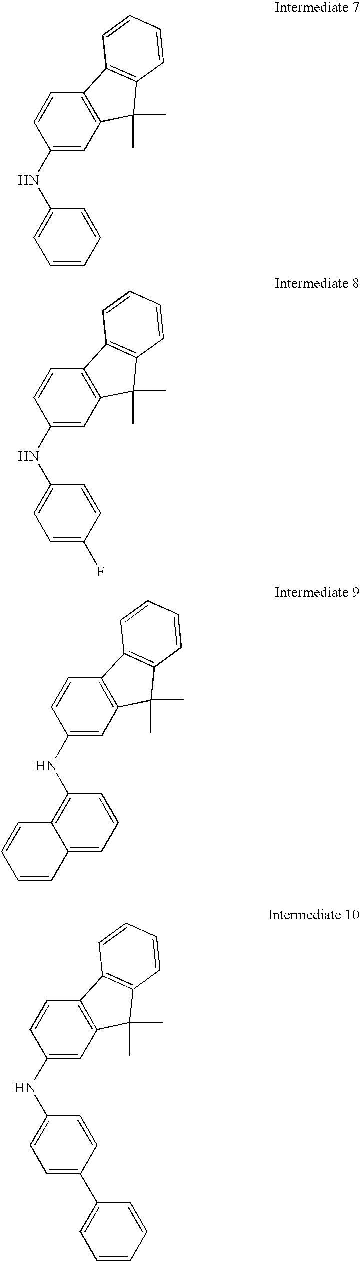

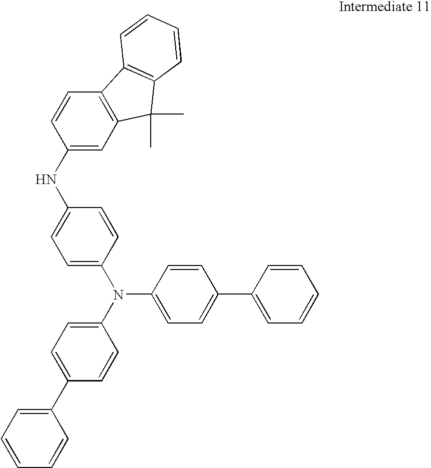

### Synthesis Example 12

### Synthesis of Intermediate 12

9.51 g (30 mmol) of Intermediate 5, 16.6 g (60 mmol) of Intermediate 1, 1.7 g (1.5 mmol) of Pd(PPh₃)₄, and 20 g (150 mmol) of K₂CO₃ were dissolved in 100 ml of a mixed solution of THF/H₂O (2:1) to form a mixture. The mixture was stirred at 80° C. for 5 hours, and a reaction was allowed to take place. The reaction mixture was extracted with 600 ml of diethyl ether three times. An organic layer was collected and dried using magnesium sulfate, and the residue obtained by evaporating the solvent was recrystallized with dichloromethane and normal hexane to obtain 10.03 g (yield 65%) of Intermediate 12. Intermediate 12 was evaluated by NMR, and the results thereof are shown below.

¹H NMR (CDCl₃, 400 MHz) δ (ppm)-8.16-8.14 (m, 1H), 7.94 (d, 1H), 7.68 (d, 1H), 7.63 (s, 1H), 7.50-7.46 (m, 5H), 7.36-7.27 (m, 5H), 7.07-6.94 (m, 4H), 1.85 (s, 6H) ¹³C NMR (CDCl₃, 100 MHz) δ (ppm)-163.3, 150.7, 144.7, 144.2, 141.6, 141.0, 137.0, 136.0, 134.0, 131.8, 131.6, 129.8, 129.3, 129.2, 127.4, 127.1, 126.8, 126.3, 121.8, 121.2, 120.6, 120.4, 119.9, 119.5, 118.6, 115.4, 114.5, 109.0, 49.6, 24.5

### Synthesis Example 13

### Synthesis of Intermediate 13

Intermediate 13 was synthesized to obtain a yield of 63% in the same manner as in the synthesis of Intermediate 12, except that Intermediate 2 was used instead of Intermediate 1. Intermediate 13 was evaluated by NMR, and the results thereof are shown below.

¹H NMR (CDCl₃, 400 MHz) δ (ppm)-8.15 (d, 1H), 7.95-7.91 (m, 2H), 7.81-7.46 (m, 9H), 7.36-7.27 (m, 4H), 7.06 (d, 1H), 7.00-6.94 (m, 4H), 1.85 (s, 6H)

¹³C NMR (CDCl₃, 100 MHz) δ (ppm)-163.3, 150.7, 144.9, 144.2, 141.6, 140.0, 136.1, 136.0, 134.0, 131.8, 131.6, 129.5, 129.3, 129.2, 128.2, 127.9, 127.6, 126.8, 126.7, 126.2, 124.3, 122.5, 122.3, 121.8, 121.4, 121.2, 120.6, 119.9, 118.6, 117.3, 116.6, 114.5, 110.2, 105.1, 49.6, 24.5

### Synthesis Example 14

### Synthesis of Intermediate 14

Intermediate 14 was synthesized to obtain a yield of 62% in the same manner as in the synthesis of Intermediate 12, except that Intermediate 3 was used instead of Intermediate 1. Intermediate 14 was evaluated by NMR, and the results thereof are shown below.

¹H NMR (CDCl₃, 400 MHz) δ (ppm)-8.16-8.14 (m, 1H), 7.95 (d, 1H), 7.69 (d, 1H), 7.63 (s, 1H), 7.56-7.46 (m, 3H), 7.36-7.27 (m, 4H), 7.08-6.94 (m, 6H), 1.85 (s, 6H)

¹³C NMR (CDCl₃, 100 MHz) δ (ppm)-163.3, 162.0, 155.5, 150.7, 144.2, 143.8, 141.6, 140.0, 136.1, 136.0, 134.0, 131.6, 129.3, 129.2, 126.8, 126.3, 125.5, 125.3, 121.8, 121.2, 120.6, 120.4, 119.9, 119.5, 118.6, 115.4, 114.5, 113.8, 113.1, 109.0, 49.6, 24.5

### Synthesis Example 15

### Synthesis of Intermediate 15

Intermediate 15 was synthesized to obtain a yield of 58% in the same manner as in the synthesis of Intermediate 12, except that Intermediate 4 was used instead of Intermediate 1. Intermediate 15 was evaluated by NMR, and the results thereof are shown below.

¹H NMR (CDCl₃, 400 MHz) δ (ppm)-8.16-8.14 (m, 1H), 7.95 (d, 1H), 7.69 (d, 1H), 7.63 (s, 1H), 7.60-7.56 (m, 2H), 7.49-7.45 (m, 1H), 7.41-7.27 (m, 9H), 7.07-6.94 (m, 4H), 6.64-6.60 (m, 2H), 1.85 (s, 6H)

¹³C NMR (CDCl₃, 100 MHz) δ (ppm)-163.3, 150.7, 144.2, 144.1, 141.6, 140.3, 139.3, 136.9, 136.0, 135.4, 134.0, 131.9, 131.8, 131.6, 129.3, 129.2, 128.9, 128.8, 127.2, 126.8, 126.3, 121.8, 121.2, 120.6, 120.4, 119.9, 119.5, 118.6, 116.8, 115.4, 114.5, 109.0, 49.6, 24.5

### Synthesis Example 16

### Synthesis of Intermediate 16

Intermediate 16 was synthesized to obtain a yield of 55% in the same manner as in the synthesis of Intermediate 12, except that Intermediate 6 was used instead of Intermediate 5. Intermediate 16 was evaluated by NMR, and the result thereof is shown below.

¹H NMR (CDCl₃, 400 MHz) δ (ppm)-8.16-8.14 (m, 1H), 7.95 (d, 1H), 7.77 (d, 1H), 7.65-7.63 (m, 2H), 7.59 (dd, 1H), 7.52-7.46 (m, 9H), 7.37-7.27 (m, 4H), 7.20-7.13 (m, 5H), 7.06 (dd, 1H), 7.02-6.97 (m, 3H)

¹³C NMR (CDCl₃, 100 MHz) δ (ppm)-164.2, 151.5, 145.4, 144.7, 142.9, 141.0, 140.7, 137.0, 135.7, 135.1, 132.8, 131.3, 130.1, 129.8, 128.8, 127.8, 127.4, 127.1, 126.3, 126.0, 125.1, 122.9, 122.3, 121.7, 120.4, 119.9, 119.5, 118.6, 115.4, 114.1, 109.0, 72.9

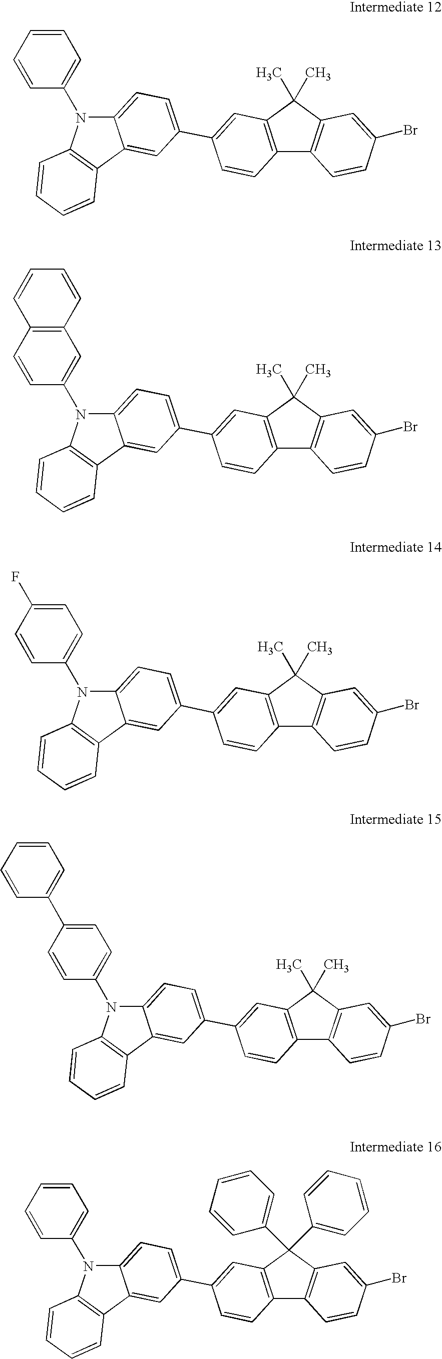

### Synthesis Example 17

### Synthesis of Compound 1

5.14 g (10 mmol) of Intermediate 12, 3.42 g (12 mmol) of Intermediate 7, 2.9 g (30 mmol) of t-BuONa, 366 mg (0.4 mmol) of Pd₂(dba)₃, and 80 mg (0.4 mmol) of P(t-Bu)₃ were dissolved in 60 ml of toluene to form a mixture. The mixture was stirred at 90° C. for 3 hours, and a reaction was allowed to take place. After the reaction was completed, the reaction mixture was cooled to room temperature, and extracted with distilled water and 50 ml of diethyl ether three times. An organic layer was collected and dried using magnesium sulfate, and the residue obtained by evaporating the solvent was separately purified by silica gel column chromatography to obtain 5.9 g (yield 82%) of Compound 1. Compound 1 was evaluated by NMR, and the results thereof are shown below.

¹H NMR (CDCl₃, 400 MHz) δ (ppm)-8.16-8.14 (m, 1H), 7.99-7.93 (m, 2H), 7.69-7.46 (m, 8H), 7.37-7.27 (m, 6H), 7.24-6.93 (m, 8H), 6.64-6.59 (m, 1H), 6.31 (dd, 2H), 5.65 (dd, 2H), 1.85 (s, 12H)

¹³C NMR (CDCl₃, 100 MHz) δ (ppm)-158.7, 148.6, 148.1, 147.1, 147.0, 144.9, 144.7, 141.0, 139.6, 138.0, 137.0, 136.5, 134.8, 134.0, 131.8, 129.8, 129.4, 129.3, 127.8, 127.4, 127.1, 126.8, 126.3, 126.1, 124.0, 122.9, 121.7, 121.1, 120.5, 120.4, 119.9, 119.5, 118.7, 118.6, 117.8, 117.2, 115.4, 114.5, 112.7, 109.0, 46.1, 44.9, 24.5

### Synthesis Example 18

### Synthesis of Compound 6

Compound 6 was synthesized to obtain a yield of 75% in the same manner as in the synthesis of Compound 1, except that Intermediate 8 was used instead of Intermediate 7. Compound 6 was evaluated by NMR, and the results thereof are shown below.

¹H NMR (CDCl₃, 400 MHz) δ (ppm)-8.16-8.14 (m, 1H), 7.99-7.93 (m, 2H), 7.69-7.46 (m, 8H), 7.37-6.93 (m, 14H), 6.73 (dd, 2H), 6.31 (dd, 2H), 1.85 (s, 12H)

¹³C NMR (CDCl₃, 100 MHz) δ (ppm)-162.0, 158.7, 155.5, 148.1, 147.1, 146.0, 144.7, 141.0, 140.7, 139.6, 138.0, 137.0, 136.5, 134.8, 134.0, 131.8, 129.8, 129.3, 127.8, 127.4, 127.1, 126.8, 126.4, 126.3, 126.2, 126.1, 121.7, 121.1, 120.5, 120.4, 119.9, 119.5, 118.7, 118.6, 117.8, 117.2, 116.2, 115.5, 115.4, 114.5, 112.7, 109.0, 46.1, 44.9, 24.5

### Synthesis Example 19

### Synthesis of Compound 7

Compound 7 was synthesized to obtain a yield of 80% in the same manner as in the synthesis of Compound 1, except that Intermediate 9 was used instead of Intermediate 7. Compound 7 was evaluated by NMR, and the results thereof are shown below.

¹H NMR (CDCl₃, 400 MHz) δ (ppm)-8.50 (dd, 1H), 8.16-8.14 (m, 1H), 7.99-7.93 (m, 2H), 7.85 (dd, 1H), 7.69-6.93 (m, 24H), 6.29 (dd, 2H), 6.08 (dd, 1H), 1.85 (s, 12H)

¹³C NMR (CDCl₃, 100 MHz) δ (ppm)-158.7, 150.6, 150.5, 150.0, 147.1, 144.7, 141.0, 139.6, 138.0, 137.0, 136.5, 134.8, 134.0, 131.8, 129.8, 129.3, 128.6, 127.8, 127.6, 127.4, 127.1, 126.8, 126.7, 126.3, 126.1, 126.0, 124.1, 123.5, 122.9, 121.7, 121.5, 120.5, 120.4, 119.9, 119.5, 118.7, 118.6, 118.0, 117.4, 116.4, 115.4, 114.5, 112.9, 109.0, 46.1, 44.9, 24.5

### Synthesis Example 20

### Synthesis of Compound 9

Compound 9 was synthesized to obtain a yield of 77% in the same manner as in the synthesis of Compound 1, except that Intermediate 10 was used instead of Intermediate 7. Compound 9 was evaluated by NMR, and the results thereof are shown below.

¹H NMR (CDCl₃, 400 MHz) δ (ppm)-8.16-8.14 (m, 1H), 7.99-7.93 (m, 2H), 7.76-6.93 (m, 27H), 6.43-6.29 (m, 4H), 1.85 (s, 12H)

¹³C NMR (CDCl₃, 100 MHz) δ (ppm)-158.7, 148.6, 148.1, 147.1, 146.3, 145.5, 144.7, 141.0, 139.6, 138.0, 137.0, 136.9, 136.5, 135.4, 134.8, 134.2, 134.0, 131.8, 129.8, 129.3, 128.9, 128.8, 127.7, 127.4, 127.2, 127.1, 126.8, 126.3, 126.1, 117.8, 117.7, 117.2, 115.4, 114.5, 112.7, 109.0, 46.1, 44.9, 24.5

### Synthesis Example 21

### Synthesis of Compound 22

Compound 22 was synthesized to obtain a yield of 72% in the same manner as in the synthesis of Compound 1, except that Intermediate 11 was used instead of Intermediate 7. Compound 22 was evaluated by NMR, and the results thereof are shown below.

¹H NMR (CDCl₃, 400 MHz) δ (ppm)-8.16-8.14 (m, 1H), 7.99-7.93 (m, 2H), 7.76-7.63 (m, 10H), 7.56 (dd, 2H), 7.49 (dd, 4H), 7.41-7.07 (m, 15H), 7.00-6.93 (m, 3H), 6.47-6.43 (m, 4H), 6.32-6.19 (m, 6H), 1.85 (s, 12H) ¹³C NMR (CDCl₃, 100 MHz) δ (ppm)-158.7, 148.6, 148.1, 147.1, 146.8, 145.7, 144.7, 143.8, 141.0, 139.6, 138.0, 137.0, 136.9, 136.5, 135.4, 134.8, 134.2, 134.0, 131.8, 129.8, 129.3, 128.9, 128.8, 127.8, 127.4, 127.2, 127.1, 126.8, 126.3, 126.1, 121.7, 121.1, 120.5, 119.9, 119.7, 119.6, 118.7, 117.8, 117.7, 117.2, 115.4, 114.5, 112.7, 109.0, 46.1, 44.9, 24.5

### Synthesis Example 22

### Synthesis of Compound 46

Compound 46 was synthesized to obtain a yield of 75% in the same manner as in the synthesis of Compound 9, except that Intermediate 13 was used instead of Intermediate 12. Compound 46 was evaluated by NMR, and the results thereof are shown below.

¹H NMR (CDCl₃, 400 MHz) δ (ppm)-8.15 (d, 1H), 7.99-7.91 (m, 3H), 7.80-7.49 (m, 13H), 7.41-7.07 (m, 11H), 7.00-6.93 (m, 3H), 6.43-6.39 (m, 2H), 6.31 (dd, 2H), 1.85 (s, 12H)

¹³C NMR (CDCl₃, 100 MHz) δ (ppm)-158.7, 148.6, 148.1, 147.1, 146.3, 145.5, 144.9, 140.0, 139.6, 138.0, 137.0, 136.9, 136.5, 136.1, 135.4, 134.8, 134.2, 134.0, 131.8, 129.5, 129.3, 128.9, 128.8, 128.2, 127.8, 127.6, 127.2, 126.8, 126.7, 126.2, 126.1, 124.3, 122.5, 122.3, 121.7, 121.4, 121.1, 120.5, 119.9, 118.7, 118.6, 117.8, 117.7, 117.3, 117.2, 116.6, 114.5, 112.7, 110.2, 105.1, 46.1, 44.9, 24.5

### Synthesis Example 23

### Synthesis of Compound 60

Compound 60 was synthesized to obtain a yield of 78% in the same manner as in the synthesis of Compound 9, except that Intermediate 14 was used instead of Intermediate 12. Compound 60 was evaluated by NMR, and the results thereof are shown below.

¹H NMR (CDCl₃, 400 MHz) δ (ppm)-8.16-8.14 (m, 1H), 7.99-7.93 (m, 2H), 7.76-7.52 (m, 10H), 7.41-6.93 (m, 16H), 6.43-6.39 (m, 2H), 6.31 (dd, 2H), 1.85 (s, 12H)

¹³C NMR (CDCl₃, 100 MHz) δ (ppm)-161.9, 158.7, 155.5, 148.6, 148.1, 147.1, 146.3, 145.5, 143.8, 140.0, 139.6, 138.0, 137.0, 136.9, 136.5, 136.1, 135.4, 134.8, 134.2, 134.0, 131.8, 129.3, 128.9, 128.8, 127.8, 127.2, 126.8, 126.3, 126.1, 125.5, 125.3, 121.7, 121.1, 120.5, 120.4, 119.9, 119.5, 118.7, 118.6, 117.8, 117.7, 117.2, 115.4, 114.5, 113.8, 113.1, 112.7, 109.0, 46.1, 44.9, 24.5

### Synthesis Example 24

### Synthesis of Compound 72

Compound 72 was synthesized to obtain a yield of 81% in the same manner as in the synthesis of Compound 7, except that Intermediate 15 was used instead of Intermediate 12. Compound 72 was evaluated by NMR, and the results thereof are shown below.

¹H NMR (CDCl₃, 400 MHz) δ (ppm)-8.50 (dd, 1H), 8.16-8.14 (m, 1H), 7.99-7.93 (m, 2H), 7.85 (dd, 1H), 7.69-6.93 (m, 26H), 6.64-6.60 (m, 2H), 6.29 (dd, 2H), 6.09 (dd, 1H), 1.85 (s, 12H)

¹³C NMR (CDCl₃, 100 MHz) δ (ppm)-158.7, 150.6, 150.5, 150.0, 147.1, 144.1, 140.3, 139.6, 139.3, 138.0, 137.0, 136.9, 136.5, 135.4, 134.8, 134.0, 131.8, 129.3, 128.9, 128.8, 128.6, 127.8, 127.6, 127.2, 126.8, 126.7, 126.3, 126.1, 126.0, 124.1, 123.5, 122.9, 121.7, 121.5, 120.5, 120.4, 119.9, 119.5, 118.7, 109.0, 46.1, 44.9, 24.5

### Synthesis Example 25

### Synthesis of Compound 74

Compound 74 was synthesized to obtain a yield of 83% in the same manner as in the synthesis of Compound 9, except that Intermediate 15 was used instead of Intermediate 12. Compound 74 was evaluated by NMR, and the results thereof are shown below.

¹H NMR (CDCl₃, 400 MHz) δ (ppm)-8.16-8.14 (m, 1H), 7.99-7.93 (m, 2H), 7.76-7.53 (m, 10H), 7.41-6.93 (m, 18H), 6.64-6.60 (m, 2H), 6.43-6.39 (m, 2H), 6.31 (dd, 2H), 1.85 (s, 12H)

¹³C NMR (CDCl₃, 100 MHz) δ (ppm)-158.7, 148.6, 148.1, 147.1, 146.3, 145.5, 144.1, 140.3, 139.6, 139.3, 138.0, 137.0, 136.9, 136.5, 135.4, 134.8, 134.2, 134.0, 131.8, 129.3, 128.9, 128.8, 127.8, 127.2, 126.8, 126.3, 126.1, 121.7, 121.1, 120.5, 119.9, 119.5, 118.7, 118.6, 117.8, 117.7, 117.2, 116.8, 115.4, 114.5, 112.7, 109.0, 46.1, 44.9, 24.5

### Synthesis Example 26

### Synthesis of Compound 83

Compound 83 was synthesized to obtain a yield of 69% in the same manner as in the synthesis of Compound 1, except that Intermediate 16 was used instead of Intermediate 12. Compound 83 was evaluated by NMR, and the results thereof are shown below.

¹H NMR (CDCl₃, 400 MHz) δ (ppm)-8.16-8.14 (m, 1H), 7.99-7.93 (m, 2H), 7.69-7.56 (m, 10H), 7.49 (d, 4H), 7.37-6.93 (m, 18H), 6.64-6.59 (m, 1H), 6.42 (dd, 1H), 6.31 (dd, 1H), 5.67-5.63 (m, 2H), 1.85 (s, 6H)

¹³C NMR (CDCl₃, 100 MHz) δ (ppm)-159.7, 149.5, 148.1, 147.1, 146.5, 144.7, 144.4, 141.0, 140.8, 138.3, 138.0, 137.0, 136.5, 136.2, 135.1, 134.5, 132.8, 129.8, 129.4, 128.8, 128.6, 127.8, 127.4, 127.1, 126.8, 126.3, 126.1, 126.0, 125.1, 124.0, 122.9, 122.2, 121.7, 120.4, 119.9, 119.5, 118.7, 118.6, 117.8, 116.9, 115.4, 114.1, 113.6, 112.7, 109.0, 69.5, 44.9, 24.5

### Example 1

An anode was prepared by cutting a 15 Ωcm² (1200 Å) ITO Corning™ glass substrate into a size of 50 mm×50 mm×0.7 mm, ultrasonic cleaning the substrate using isopropyl alcohol and pure water for 5 minutes each, and then irradiating the substrate under UV light for 30 minutes and exposing it to ozone to clean. The prepared anode was then installed in a vacuum deposition apparatus.

2-TNATA, was vacuum-deposited on the anode to a thickness of 600 Å to form a HIL, and Compound 1 as a hole transporting compound was vacuum-deposited on the HIL to a thickness of 300 Å to form a HTL.

A green fluorescent host Alq₃ and a green fluorescent dopant C545T were deposited simultaneously with a weight ratio of 98:2 on the HTL to form an EML with a thickness of 300 Å.

Next, Alq₃ was deposited on the EML to a thickness of 300 Å to form an ETL, and LiF was deposited to a thickness of 10 Å on the ETL to form an EIL. Finally, Al was vacuum-deposited on the EIL to a thickness of 3000 Å to form a LiF/Al electrode (cathode), thereby completing the manufacture of an organic light emitting device.

The device had a driving voltage of 6.42 V, a high emission brightness of 8,303 cd/m², color coordinates of (0.310, 0.644), and an emission efficiency of 16.6 cd/A, at a current density of 50 mA/cm².

### Example 2

An organic light emitting device was manufactured in the same manner as in Example 1, except that Compound 6 was used instead of Compound 1 in forming the HTL.

The device had a driving voltage of 6.28 V, a high emission brightness of 8,485 cd/m², color coordinates of (0.310, 0.643), and an emission efficiency of 17.0 cd/A, at a current density of 50 mA/cm².

### Example 3

An organic light emitting device was manufactured in the same manner as in Example 1, except that Compound 7 was used instead of Compound 1 in forming the HTL.

The device had a driving voltage of 6.16 V, a high emission brightness of 8,590 cd/m², color coordinates of (0.309, 0.642), and an emission efficiency of 17.2 cd/A, at a current density of 50 mA/cm².

### Example 4

An organic light emitting device was manufactured in the same manner as in Example 1, except that Compound 9 was used instead of Compound 1 in forming the HTL.

The device had a driving voltage of 6.45 V, a high emission brightness of 7,634 cd/m², color coordinates of (0.310, 0.642), and an emission efficiency of 15.3 cd/A, at a current density of 50 mA/cm².

### Example 5

An organic light emitting device was manufactured in the same manner as in Example 1, except that Compound 22 was used instead of Compound 1 in forming the HTL.

The device had a driving voltage of 6.34 V, a high emission brightness of 8,036 cd/m², color coordinates of (0.309, 0.641), and an emission efficiency of 16.1 cd/A, at a current density of 50 mA/cm².

### Example 6

An organic light emitting device was manufactured in the same manner as in Example 1, except that Compound 46 was used instead of Compound 1 in forming the HTL.

The device had a driving voltage of 6.39 V, a high emission brightness of 8,126 cd/m², color coordinates of (0.310, 0.643), and an emission efficiency of 16.3 cd/A, at a current density of 50 mA/cm².

### Example 7

An organic light emitting device was manufactured in the same manner as in Example 1, except that Compound 60 was used instead of Compound 1 in forming the HTL.

The device had a driving voltage of 6.29 V, a high emission brightness of 7,914 cd/m², color coordinates of (0.311, 0.644), and an emission efficiency of 15.8 cd/A, at a current density of 50 mA/cm².

### Example 8

An organic light emitting device was manufactured in the same manner as in Example 1, except that Compound 72 was used instead of Compound 1 in forming the HTL.

The device had a driving voltage of 6.24 V, a high emission brightness of 8,393 cd/m², color coordinates of (0.309, 0.643), and an emission efficiency of 16.8 cd/A, at a current density of 50 mA/cm².

### Example 9

An organic light emitting device was manufactured in the same manner as in Example 1, except that Compound 74 was used instead of Compound 1 in forming the HTL.

The device had a driving voltage of 6.30 V, a high emission brightness of 8,090 cd/m², color coordinates of (0.309, 0.641), and an emission efficiency of 16.2 cd/A, at a current density of 50 mA/cm².

### Example 10

An organic light emitting device was manufactured in the same manner as in Example 1, except that Compound 83 was used instead of Compound 1 in forming the HTL.

The device had a driving voltage of 6.34 V, a high emission brightness of 7,898 cd/m², color coordinates of (0.310, 0.642), and an emission efficiency of 15.8 cd/A, at a current density of 50 mA/cm².

### Comparative Example 1

An organic light emitting device was manufactured in the same manner as in Example 1, except that 4,4′-bis[N-(1-naphthyl)-N-phenylamino]biphenyl (NPB) was used instead of Compound 1 in forming the HTL.

The device had a driving voltage of 7.45 V, a high emission brightness of 6,102 cd/m², color coordinates of (0.309, 0.642), and an emission efficiency of 12.2 cd/A, at a current density of 50 mA/cm².

Each of the organic light emitting devices of Examples 1 through 10 had a driving voltage of about at least 1 V lower than the organic light emitting device of Comparative Example 1, and improved efficiency, as well as excellent I-V-L characteristics.

Half life-span values of the organic light emitting devices of Examples 1, 3, 4, 6, 7, and 9 and Comparative Example 1 are shown in Table 1 below:

TABLE 1

| | Hole transporting | Half life-span |
|---|---|---|
| | material | (hr @100 mA/cm²) |
| Example 1 | Compound 1 | 268 hr |
| Example 3 | Compound 7 | 291 hr |
| Example 4 | Compound 9 | 258 hr |
| Example 6 | Compound 46 | 295 hr |
| Example 7 | Compound 60 | 353 hr |
| Example 9 | Compound 74 | 321 hr |
| Comparative | NPB | 113 hr |
| Example 1 | | |

Referring to Table 1, it can be seen that the organic light emitting devices of Examples 1, 3, 4, 6, 7, and 9 have improved life-span compared with the organic light emitting device of Comparative Example 1.

As described above, the amine-based compound represented by Formula 1 has excellent electrical properties and charge transporting capability, and thus is suitable for use as a hole injecting material, hole transporting material, and/or a light emitting material of an organic light emitting device.

While the present invention has been described with reference to certain exemplary embodiments, it will be understood by those of ordinary skill in the art that various changes may be made to the described embodiments without departing from the spirit and scope of the present invention, as defined in the appended claims and their equivalents.

What is claimed is:

1. An amine-based compound represented by Formula 1, 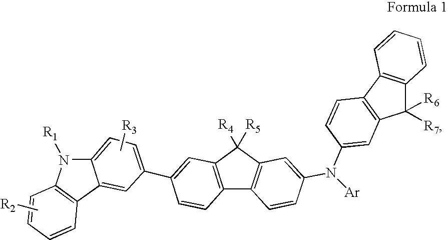
wherein Ar is a substituted or unsubstituted C₆-C₃₀ aryl group, a substituted or unsubstituted C₆-C₂₀ aryloxy group, a substituted or unsubstituted C₄-C₂₀ heteroaryl group, or a substituted or unsubstituted C₄-C₂₀ condensed polycyclic group,
R₁ is hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ heteroaryl group, or a substituted or unsubstituted C₄-C₂₀ condensed polycyclic group,
each of R₂ and R₃ is independently hydrogen, a substituted or unsubstituted C₁-C₂₀ alkyl group, a substituted or unsubstituted C₁-C₂₀ alkoxy group, a substituted or unsubstituted C₆-C₃₀ aryl group, a substituted or unsubstituted C₄-C₂₀ condensed polycyclic group, fluorine, a cyano group, or an amino group, and
each of R₄, R₅, R₆, and R₇ is independently hydrogen, a substituted or unsubstituted C₁-C₂₀ alkyl group, a substituted or unsubstituted C₁-C₂₀ alkoxy group, or a substituted or unsubstituted C₆-C₃₀ aryl group.

2. The amine-based compound of claim 1, wherein Ar is a substituted or unsubstituted C₆-C₁₆ aryl group, or a substituted or unsubstituted C₄-C₁₆ heteroaryl group.

3. The amine-based compound of claim 1, wherein Ar is a phenyl group, a biphenyl group, a terphenyl group, a naphthyl group, a pentalenyl group, an indenyl group, an anthracenyl group, an azulenyl group, a heptalenyl group, an acenaphthylenyl group, a fluorenyl group, a penanthrenyl group, a triphenylenyl group, a naphthacenyl group, a phenalenyl group, a pyrenyl group, a pentaphenyl group, a hexaphenyl group, a pyridinyl group, a quinolinyl group, a pyrazinyl group, a pyrimidinyl group, or a carbazolyl group, each of which may be unsubstituted or substituted with a halogen atom, a cyano group, an amino group, a (C₁-C₁₀ alkyl)amino group, a di(C₁-C₁₀ alkyl)amino group, a (C₆-C₁₄ aryl)amino group, a di(C₆-C₁₄ aryl)amino group, a C₁-C₁₀ alkyl group, a C₁-C₁₀ alkoxy group, a C₆-C₁₄ aryloxy group, a C₆-C₁₄ aryl group, or a C₆-C₁₄ aryl group substituted with a C₆-C₁₄ aryl substituent.

4. The amine-based compound of claim 1, wherein Ar is represented by any of Formulae 2a through 2q: 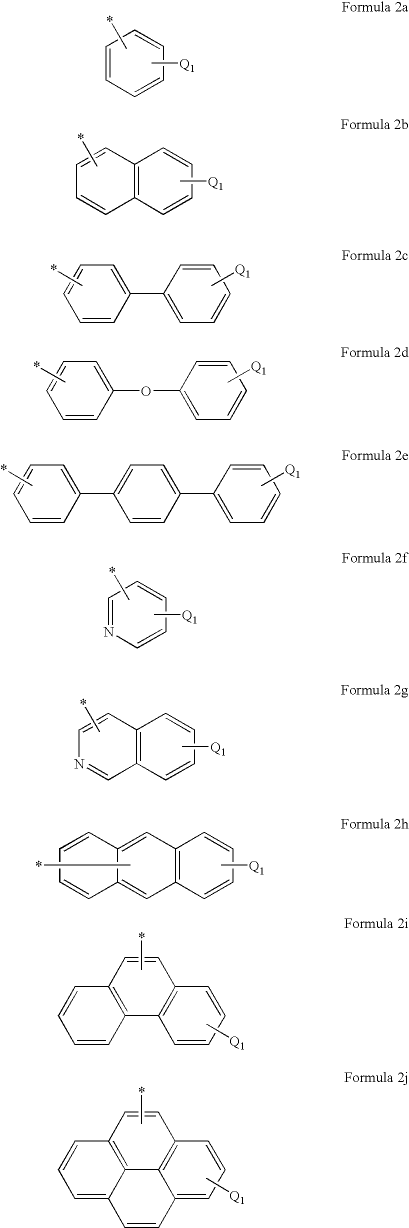 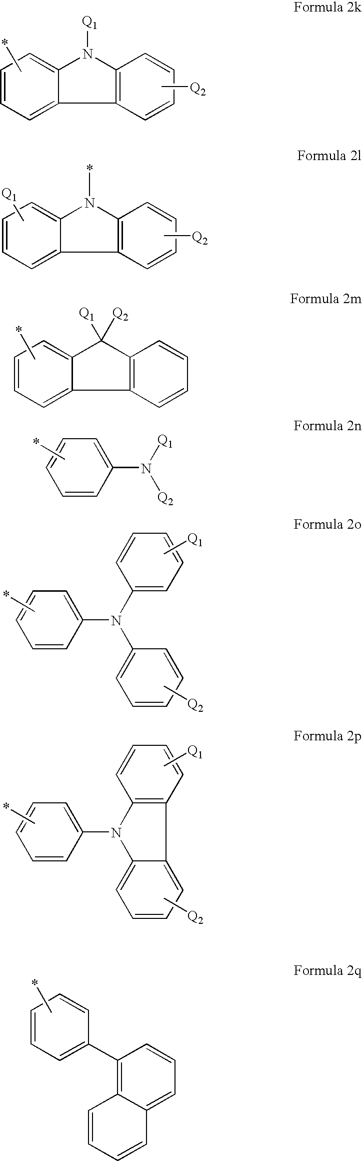
wherein each of Q₁ and Q₂ is independently hydrogen, fluorine, a cyano group, an amino group, a C₁-C₁₀ alkyl group, a C₁-C₁₀ alkoxy group, or a C₆-C₁₄ aryl group, and * represents a binding site with the N of Formula 1.

5. The amine-based compound of claim 1, wherein R₁ is a substituted or unsubstituted C₆-C₁₄ aryl group.

6. The amine-based compound of claim 1, wherein R₁ is a phenyl group, a naphthyl group, a pentalenyl group, an indenyl group, an anthracenyl group, an azulenyl group, a heptalenyl group, an acenaphthylenyl group, a fluorenyl group, a penanthrenyl group, a triphenylenyl group, a naphthacenyl group, a phenalenyl group, a pyrenyl group, a pentaphenyl group, or a hexaphenyl group, each of which may be unsubstituted or substituted with a halogen atom, a cyano group, an amino group, a (C₁-C₁₀ alkyl)amino group, a di(C₁-C₁₀ alkyl)amino group, a (C₆-C₁₄ aryl)amino group, a di(C₆-C₁₄ aryl)amino group, a C₁-C₁₀ alkyl group, a C₁-C₁₀ alkoxy group, a C₆-C₁₄ aryloxy group, a C₆-C₁₄ aryl group, or a C₆-C₁₄ aryl group substituted with a C₆-C₁₄ aryl substituent.

7. The amine-based compound of claim 1, wherein R₁ is a phenyl group, a naphthyl group, a halophenyl group, or a phenyl group substituted with a phenyl substituent.

8. The amine-based compound of claim 1, wherein each of R₂ and R₃ is independently hydrogen, fluorine, a cyano group, an amino group, a C₁-C₁₀ alkyl group, or a C₁-C₁₀ alkoxy group.

9. The amine-based compound of claim 1, wherein each of R₄ through R₇ is independently hydrogen, a C₁-C₁₀ alkyl group, a C₁-C₁₀ alkoxy group, or a C₆-C₁₄ aryl group.

10. The amine-based compound of claim 1, wherein Formula 1 is represented by any one of the following formulae:

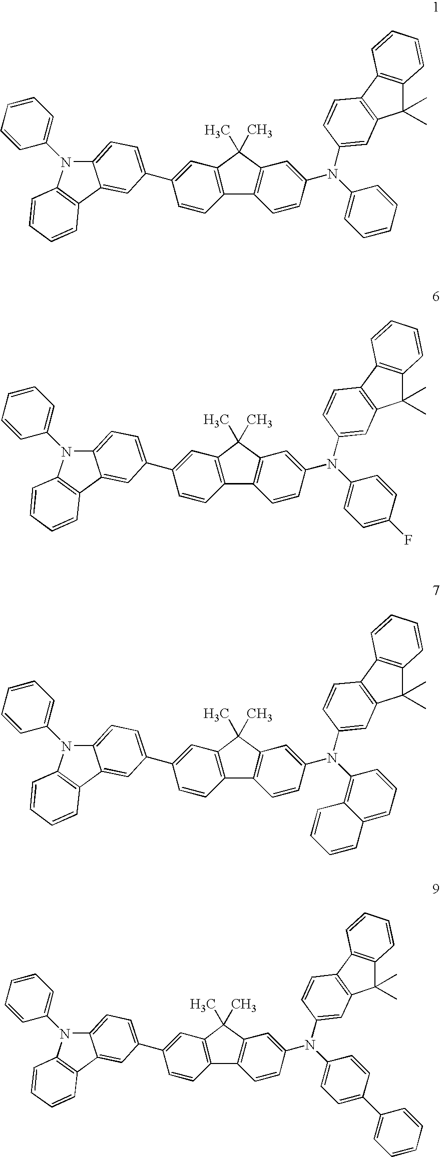

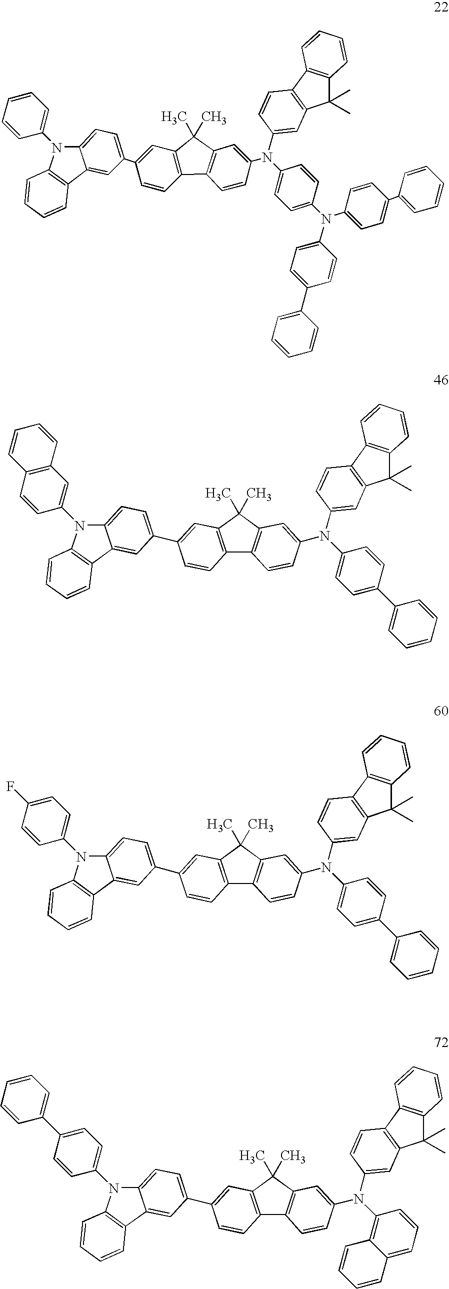

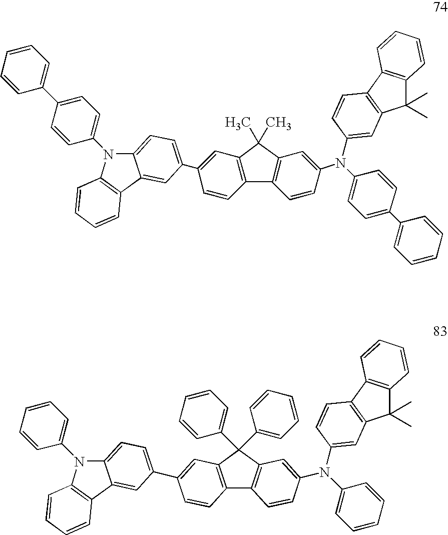

11. An organic light emitting device comprising: a first electrode; a second electrode; and an organic film between the first electrode and the second electrode, wherein the organic film comprises the amine-based compound according to claim 1.

12. The organic light emitting device of claim 11, wherein the organic film comprises a hole injection layer or a hole transport layer.

13. The organic light emitting device of claim 11, wherein the organic film comprises a single film having both hole injection and hole transport capabilities.

14. The organic light emitting device of claim 11, wherein the organic film comprises an emissive layer.

15. The organic light emitting device of claim 11, wherein the organic film further comprises a layer selected from the group consisting of hole injection layers, hole transport layers, electron blocking layers, emissive layers, hole blocking layers, electron transport layers, electron injection layers, and combinations thereof.

16. The organic light emitting device of claim 15, wherein the organic light emitting device has a first electrode/hole injection layer/emissive layer/second electrode structure, a first electrode/hole injection layer/hole transport layer/emissive layer/electron transport layer/second electrode structure, or a first electrode/hole injection layer/hole transport layer/emissive layer/electron transport layer/electron injection layer/second electrode structure.

17. A flat panel display device comprising the organic light emitting device of claim 11 and a thin-film transistor, wherein the first electrode of the organic light emitting device is electrically connected to a source electrode or a drain electrode of the thin-film transistor.
